(19) United States
(12) Patent Application Publication
(10) Pub. No.: US 2019/0302133 A1
(43) Pub. Date: Oct. 3, 2019

(54) Method for Evaluating Health of Skin

(71) Applicant: KAO CORPORATION, Chuo-ku, Tokyo (JP)

(72) Inventors: Junko ISHIKAWA, Utsunomlya-shi, Tochigi (JP); Urara YOKOSE, Utsunomlya-shi, Tochigi (JP); Kyoko SHIMA, Saitama-shi,Saitama (JP); Yuki MOROKUMA, Funabashi-shi, Chiba (JP)

(73) Assignee: KAO CORPORATION, Chuo-ku, Tokyo (JP)

(21) Appl. No.: 16/075,822

(22) PCT Filed: Dec. 27, 2016
(86) PCT No.: PCT/JP2016/089003

§ 371 Date: Jan. 17, 2019

(30) Foreign Application Priority Data
Mar. 30, 2016 (JP) 2016-067643

(51) Int. Cl. G01N 33/92; G01N 30/72; G16C 20/90

(52) U.S. Cl. CPC G01N 33/92; G01N 30/7233; G16C 20/90; G01N 2800/207; G01N 2800/202; G01N 2800/205; G01N 2405/08

(57) ABSTRACT

A method of evaluating the health of skin, containing the steps of:
preparing a lipid sample from a collected sample of a stratum corneum of a test subject;
quantitatively determining respective contents of one kind of ceramide component A and one kind of ceramide component B included in the prepared lipid sample prepared from a collected sample of a stratum corneum of the test subject;
calculating the content ratio of the quantitatively determined content of the ceramide component A to the quantitatively determined content of the ceramide component B; and
evaluating the health of the skin of the test subject from the calculated content ratio;
   wherein the ceramide component A is selected from the group consisting of a non-hydroxyacyl-phytosphingosine ceramide, a non-hydroxyacyl-6-hydroxysphingosine ceramide component, an esterified ω-hydroxyacyl-6-hydroxysphingosine ceramide component and an esterified ω-hydroxyacyl-phytosphingosine ceramide component; and the ceramide component B is selected from the group consisting of a non-hydroxyacyl-sphingosine ceramide and an α-hydroxyacyl-sphingosine ceramide component.

### TECHNICAL FIELD

The present invention relates to a method of evaluating health of skin, and an apparatus for evaluating health of skin.

### BACKGROUND OF THE INVENTION

Health conditions of the skin, such as presence of atopic dermatitis or psoriasis, wrinkles, slackening and other changes in the skin caused by aging, skin color and luster, state of skin blood flow, skin moistness, and degree of skin roughness or oiliness, can be judged and evaluated to a certain extent by visually examining the skin. Needless to say, however, skin health cannot be scientifically evaluated at the molecular level through a judgment of the external appearance of the skin.

Lipids in the stratum corneum are known as skin components that are involved in skin barrier function and water retention function at the molecular level and greatly affect the health of the skin. Here, the term “lipids” refers to biological molecules having long-chain fatty acid or hydrocarbon chains, and include fatty acids, glycerides, wax esters, sphingolipids, phospholipids, cholesterol, and the like. Among them, ceramides, which constitute a class of sphingolipids, are lipids that play a significant role in the health of the skin, and it has been suggested that decrease of certain kinds of ceramides is related to skin troubles such as atopic dermatitis and psoriasis.

Therefore, the ability to analyze lipids of the stratum corneum, particularly ceramides, in detail, and thereby obtain information about the types and amounts of ceramides present in the stratum corneum, can be expected to make it possible to scientifically evaluate whether the skin is healthy.

Known methods for evaluating skin health by analyzing lipids contained in a biological sample include methods of separating lipids by liquid chromatography, ionizing the lipids thus separated, detecting compositional information of various lipid molecules included in the biological sample using a mass analysis apparatus, and evaluating the health of the skin based on the compositional information of the various lipid molecules thus detected (see, for example, Patent Literatures 1 and 2, and Non-Patent Literatures 1 and 2). For example, in Patent Literature 1, as the compositional information of the lipid molecules included in a biological sample of a test subject, the amount, content ratios, average chain length, and the like are used as indicators for evaluating atopic dermatitis, psoriasis, and dry skin condition.

At the 40 Convention of Japanese Cosmetic Science Society, the inventors of the present invention reported that the content ratios of particular ceramide classes included in the stratum corneum, namely, a non-hydroxyacyl-phytosphingosine ceramide component and a non-hydroxyacyl-sphingosine ceramide component, exhibit meaningful correlations with the indices of skin quality, such as the transepidermal water loss (TEWL), the horny layer water content, the desquamation score, the skin texture score, the L* value, and the a* value. The content ratios of particular ceramide classes can therefore serve as indices for skin quality evaluation (Non-Patent Literature 3).

### CITATION LIST

### Patent Literature

Patent Literature 1: JP-A-2008-261741 (“JP-A” means unexamined published Japanese patent application)
Patent Literature 2: JP-A-2007-108060

### Non-Patent Literature

Non-Patent Literature 1: Yoshinori Masukawa, et al., Journal of Lipid Research, 2008, vol. 49, p. 1466-1476
Non-Patent Literature 2: Jeroen van Smeden, et al., Journal of Lipid Research, 2011, vol. 52, p. 1211-1221
Non-Patent Literature 3: The 40^{th} Convention of Japanese Cosmetic Science Society, Proceedings of Lectures, Subjects for General Studies, 2^{nd} page (Study I-R01), June 2015

### DISCLOSURE OF INVENTION

The present invention relates to a method of evaluating the health of skin, containing the steps of:

quantitatively determining respective contents of a non-hydroxyacyl-phytosphingosine ceramide (hereinafter, also simply referred to as “NP”) component and a non-hydroxyacyl-sphingosine ceramide (hereinafter, also simply referred to as “NS”) component included in a lipid sample prepared from a collected sample of a stratum corneum of a test subject;

calculating the content ratio of the quantitatively determined content of the NP component to the quantitatively determined content of the NS component; and

evaluating the skin condition for skin disease of the test subject from the calculated content ratio.

The present invention also relates to a method of evaluating the health of skin, containing the steps of:

quantitatively determining respective contents of one kind of ceramide component A and one kind of ceramide component B included in a lipid sample prepared from a collected sample of a stratum corneum of a test subject;

calculating the content ratio of the quantitatively determined content of the ceramide component A to the quantitatively determined content of the ceramide component B; and

evaluating the health of the skin of the test subject from the calculated content ratio;

wherein the ceramide component A is selected from the group consisting of a NP component, a non-hydroxyacyl-6-hydroxysphingosine ceramide (hereinafter, also simply referred to as “NH”) component, an esterified ω-hydroxyacyl-6-hydroxysphingosine ceramide (hereinafter, also simply referred to as “EOH”) component, and an esterified ω-hydroxyacyl-phytosphingosine ceramide (hereinafter, also simply referred to as “EOP”) component; and the ceramide component B is selected from the group consisting of a NS component and an α-hydroxyacyl-sphingosine ceramide (hereinafter, also simply referred to as “AS”) component; provided that the case of selecting the NP component as the ceramide component A and the NS component as the ceramide component B is excluded.

The present invention also relates to an apparatus for evaluating the health of skin, containing:

a quantitative determination means for quantitatively determining respective contents of a NP component and a NS component included in a lipid sample prepared from a collected sample of a stratum corneum; and

an arithmetic means for calculating the content ratio of the quantitatively determined content of the NP component to the quantitatively determined content of the NS component, and evaluating the skin condition for skin disease of a test subject from the calculated content ratio.

The present invention also relates to an apparatus for evaluating the health of skin, containing:

a quantitative determination means for quantitatively determining respective contents of one kind of ceramide component A and one kind of ceramide component B included in a lipid sample prepared from a collected sample of a stratum corneum; and

an arithmetic means for calculating the content ratio of the quantitatively determined content of the ceramide component A to the quantitatively determined content of the ceramide component B, and evaluating the health of the skin of a test subject from the calculated content ratio;

wherein the ceramide component A is selected from the group consisting of a NP component, a NH component, an EOH component, and an EOP component; and the ceramide component B is selected from the group consisting of a NS component and an AS component; provided that the case of selecting the NP component as the ceramide component A and the NS component as the ceramide component B is excluded.

Other and further features and advantages of the invention will appear more fully from the following description, appropriately referring to the accompanying drawings.

### BRIEF DESCRIPTION OF THE DRAWINGS

FIG. 1 is a block diagram schematically illustrating the configuration of an example of the apparatus for evaluating the health of the skin of the present invention.

FIG. 2 is a set of photographs substituted for drawing for the skin texture score scale used for the measurement of the skin texture score in Example 3.

FIGS. 3(a) to 3(f) are graphs obtained by plotting the skin quality scores and various ceramide content ratios measured in Example 3. FIG. 3(a) is a graph obtained by plotting the transepidermal water loss (TEWL) and the NH/NS ratio. FIG. 3(b) is a graph obtained by plotting the horny layer water content (Capacitance) and the EOH/NS ratio. FIG. 3(c) is a graph obtained by plotting the desquamation score and the EOP/NS ratio. FIG. 3(d) is a graph obtained by plotting the skin texture score and the NH/NS ratio. FIG. 3(e) is a graph obtained by plotting the L* value and the NP/AS ratio. FIG. 3(f) is a graph obtained by plotting the a* value and the NP/AS ratio.

### MODE FOR CARRYING OUT THE INVENTION

In the methods described in Patent Literatures 1 and 2 and Non-Patent Literatures 1 and 2, ceramide molecules in the stratum corneum are comprehensively analyzed, and an evaluation of the health of the skin is carried out based on the compositional information of the various ceramide molecules thus detected. The methods described in Patent Literatures 1 and 2 and Non-Patent Literatures 1 and 2 are therefore able to accurately evaluate skin health.

However, in the methods described in Patent Literatures 1 and 2 and Non-Patent Literatures 1 and 2, all species of ceramide molecules in the stratum corneum need to be analyzed comprehensively by mass analysis. Thus, development of a method of evaluating skin health more conveniently and accurately has been desired.

The present invention provides a method of evaluating the health of skin, specifically a method of conveniently and accurately evaluating the health of skin protecting the body.

The present invention also provides an apparatus for evaluating the health of skin, the apparatus being capable of conveniently and accurately evaluating the health of the skin that protects the body and being suitably usable in the method of evaluating the health of the skin mentioned above.

The inventors of the present invention conducted a thorough investigation.

As a result, the inventors found that among the ceramides present in the stratum corneum of a test subject who exhibits symptoms of a skin disease, particular ceramide classes have a bearing on skin health condition, including skin condition in skin diseases such as atopic dermatitis and psoriasis, the skin barrier function, and skin quality factors such as the horny layer water content, the skin color or skin luster, and other skin characteristics. Furthermore, the inventors found that the abundance ratio of two specific ceramide classes present in the stratum corneum is very important for the health of the skin. The inventors also found that the health of the skin can be accurately evaluated by quantitatively determining two specific ceramide classes in the stratum corneum, calculating the ratio of the quantitative values of the two ceramide classes, and performing evaluation based on the calculated quantitative ratio of the ceramide classes.

The present invention was completed based on these findings.

In the method of evaluating the health of the skin of the present invention, an evaluation of the health of the skin is performed based on the content ratio of two specific ceramide classes in the stratum corneum. Therefore, the method of evaluating the health of the skin of the present invention is more convenient to perform than conventional evaluation methods that comprehensively analyze all species of ceramide molecules in the stratum corneum. Furthermore, the method enables accurate evaluation of skin health.

Furthermore, the apparatus for evaluating the health of the skin of the present invention is capable of evaluating the health of the skin conveniently and accurately. Furthermore, the apparatus for evaluating the health of the skin of the present invention can be suitably used in the method of evaluating the health of the skin described above.

The “mass analysis” according to the present specification is a concept including calculating the quantitative value of a substance as an object of measurement into an absolute value or a relative value and performing an analysis.

In the method of evaluating the health of the skin according to the first embodiment of the present invention, an evaluation of the skin condition for skin disease of a test subject is carried out from the ratio of the amount of a NP component to the amount of a NS component, the components being included in a lipid sample prepared from a collected sample of the stratum corneum of the test subject.

In the second embodiment of the method of evaluating the health of the skin of the present invention, an evaluation of the health of the skin (skin condition for skin disease, skin quality, and the like) of a test subject is carried out from the ratio of the content of one kind of ceramide component A to the content of one kind of ceramide component B included in a lipid sample prepared from a collected sample of the stratum corneum of the test subject. Herein, the ceramide component A is selected from the group consisting of a NP component, a NH component, an EOH component, and an EOP component; and the ceramide component B is selected from the group consisting of a NS component and an AS component. However, in the second embodiment, the case of selecting the NP component as the ceramide component A and the NS component as the ceramide component B is excluded.

Furthermore, when the term “ceramide component A” is described in the present specification, this term refers to a NP component in the first embodiment, and the term refers to one kind of ceramide component selected from the group consisting of a NP component, a NH component, an EOH component, and an EOP component in the second embodiment. Furthermore, when the term “ceramide component B” is described, the term refers to a NS component in the first embodiment, and the term refers to one kind of ceramide component selected from the group consisting of a NS component and an AS component in the second embodiment.

Hereinafter, the present invention will be explained in detail with reference to the drawings.

Examples of the test subject to whom the method of the present invention are applied, include a human being, and mammals other than a human being, such as monkey, chimpanzee, dog, cat, cattle, pig, rat, and mouse.

In order to prepare a lipid sample derived from a collected sample of the stratum corneum of a test subject, the lipid sample being subjected to a mass analysis, the skin (including the scalp) or cells collected from a living body, reconstituted cells, skin tissue, and the like, can be used. Furthermore, the site from which the stratum corneum is collected can be selected as appropriate.

For example, according to the present invention, it is preferable that the site where the health of the skin is evaluated and the site where the stratum corneum is collected are the same site. Therefore, it is preferable to prepare a lipid sample from the stratum corneum collected from a site where the health of the skin needs to be evaluated or a site in the vicinity thereof.

Alternatively, it is also preferable to collect the stratum corneum from a non-rash (non-lesion) part (site where symptoms of skin disease are not seen) adjacent to a site of onset of skin disease (hereinafter, also referred to as “rash (lesion) part”), or from a healthy part of a test subject who has not developed skin disease. It is because in patients having skin disease, collection of the stratum corneum from the rash part is a quite demanding operation. Furthermore, at a non-rash part that is other than a rash part and is considered to be apparently healthy, the skin condition (presence or absence of onset, possibility of onset, progress of pathological condition, extent of healing, therapeutic effect, and the like) for skin disease can be judged. Furthermore, an analysis of the predisposition (tendency toward skin disease) in a healthy person that has not developed skin disease can be predicted even without performing a genetic analysis or an analysis of blood components. Thus, a possibility of the onset of a skin disease or the prophylactic state in a healthy person can be conveniently judged.

Furthermore, in the case of evaluating the skin quality as the health of the skin in the present invention, the site where the skin quality is evaluated and the site where the stratum corneum is collected can be selected as appropriate. It is preferable that those sites are predetermined sites on the face, and it is preferable that those sites are in the buccal region.

The method for collecting the stratum corneum from a predetermined site of a test subject can be selected as appropriate from conventional methods. For example, a method of attaching an adhesive surface of an adhesive tape to a predetermined site, subsequently peeling off the adhesive tape, and thereby collecting the stratum corneum (tape stripping method) can be preferably employed. Regarding preferred conditions for collecting the horny layer, to explain the case of using a film masking tape (manufactured by Teraoka Seisakusho Co., Ltd.) or a PPS tape (manufactured by Nichiban Co., Ltd.) as an example, a tape which measures about 2.5 cm in width×3 to 5 cm in length is attached to the skin, subsequently the tape is peeled off, and thus the horny layer is collected. Depending on the skin condition, this operation is repeated about 1 to 10 times at the same site, and the horny layer is collected in the depth direction of the skin.

In this case, the site from which the stratum corneum is collected may be subjected to a preliminary treatment for removing body hair or hair, sebum components existing on the surface, contaminants, and the like.

Regarding a method for preparing a lipid sample containing ceramides from a collected sample of the stratum corneum collected by a tape stripping method or the like, for example, it is preferable that ceramides are extracted from the collected sample using a solvent in which the solubility of ceramides is high and other component such as a tape are not likely to be dissolved. Examples of such a solvent include methanol, ethanol, and isopropanol.

Furthermore, a lipid sample can also be prepared according to a conventional method such as the Bligh and Dyer method or the Folch method. Furthermore, adhesive components of a tape or low-polar lipids may also be removed by means of a solid phase. For example, it is preferable that adhesive components of a tape or low-polar lipids are removed by using a silica gel cartridge for solid phase extraction and a solvent such as chloroform or methanol.

The method for quantitatively determining a ceramide component A and a ceramide component B included in a prepared lipid sample of a test subject can be selected as appropriate from conventional methods. Examples include a thin layer chromatography method of utilizing a silica gel plate; a gas chromatography-mass analysis method of separating a ceramide component A and a ceramide component B from a lipid sample using gas chromatography, ionizing the ceramide component A and ceramide component B thus separated, and quantitatively determining the ceramide component A and ceramide component B with a mass analysis apparatus; and a liquid chromatography-mass analysis (LC-MS) method of separating a ceramide component A and a ceramide component B from a lipid sample using liquid chromatography, ionizing the ceramide component A and ceramide component B thus separated, and quantitatively determining the ceramide component A and ceramide component B with a mass analysis apparatus. In the present invention, it is preferable that the ceramide component A and the ceramide component B are quantitatively determined by the LC-MS method.

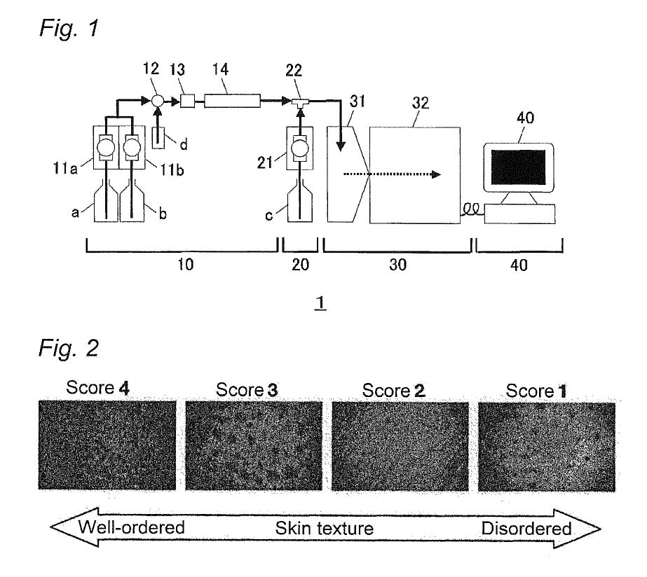

Quantitative determination of a ceramide component A and a ceramide component B can be carried out using, for example, an analysis system 1 illustrated in FIG. 1. However, the present invention is not intended to be limited to this.

The analysis system 1 illustrated in FIG. 1 is configured to include a liquid chromatograph 10, an ionization promoter liquid delivery apparatus 20, a mass analysis apparatus 30, and an arithmetic unit 40.

The liquid chromatograph 10 includes gradient pumps 11a and 11b that deliver eluents a and b, an auto-injector 12 for introducing a lipid sample solution d, a guard column 13, and a separation column 14. Here, as the lipid sample solution d, a sample solution prepared from a collected sample of the stratum corneum is used. Meanwhile, regarding the eluents a and b, eluents that are capable of appropriately retaining the group of lipid molecules such as ceramides, and separating the ceramides into different classes or different molecular species and that do not include any non-volatile acid or salt at a high concentration, are preferred. For example, it is preferable to use solutions containing a small amount of volatile formic acid or ammonium formate, as the eluents a and b. Examples of the solvent for the eluents a and b include water, methanol, ethanol, isopropanol, hexane, formic acid, ammonium formate, and mixed solvents of these. It is preferable that gradient elution is performed by using, for example, two kinds of solutions (eluent a: hexane/isopropanol/formic acid=95/5/0.1 (v/v/v); eluent b: hexane/isopropanol/50 mmol/L aqueous solution of ammonium formate=25/65/10 (v/v/v)) as the eluents a and b.

The guard column 13 is provided as necessary for the protection of the separation column 14. The guard column 13 is usually packed with the same filler as that for the separation column 14.

Regarding the filler for the guard column 13 and the separation column 14, for example, silica gel; a reverse phase column in which octadecyl groups are bonded to silica gel; or a high polar column in which diol groups, CN groups, NH₂ groups, and the like are bonded to silica gel, can be used. From the viewpoint of increasing the flow rate of the lipid sample solution flowing through the liquid chromatograph 10 and rapidly quantitatively determining the ceramide component A and the ceramide component B, the filler used for the present invention is preferably silica gel having a particle size of 3 μm or less.

The flow rate of the lipid sample solution flowing through the liquid chromatograph 10 can be appropriately set in accordance with the filler used, or the like.

By configuring the liquid chromatograph 10 as described above, the ceramide component A and the ceramide component B can be respectively separated. The ceramide component A and the ceramide component B separated by the liquid chromatograph 10 are introduced into the ionization apparatus 31 in the subsequent stage. It is preferable that the ceramide components are introduced into the ionization promoter liquid delivery apparatus 20 before that. Meanwhile, the ionization promoter liquid delivery apparatus 20 is an apparatus for promoting ionization at the ionization apparatus 31.

The ionization promoter liquid delivery apparatus 20 includes a pump 21 for delivering an ionization promoter liquid c; and a connector 22 for mixing the eluent from the separation column 14 with the ionization promoter liquid c.

The ionization promoter liquid c is usually used in order to ameliorate the problem that it is difficult to obtain sufficient ionization efficiency with an electrospray ionization (ESI) method in a case in which a low-polar solvent such as hexane is used as the eluent as described above. As the ionization promoter liquid c, a liquid that mixes satisfactorily with the eluent and has properties suitable for ionizing the eluent, such as surface tension, viscosity, ion generation ability, and solvation power, is selected as appropriate. For example, in a case in which hexane is used as the eluents a and b, a polar solvent such as isopropanol, ethanol, or methanol is preferably used as the ionization promoter liquid c.

It is preferable that a salt such as ammonium formate or ammonium acetate is added to the ionization promoter liquid c so that [M+H]⁺ or [M+H−H₂O]⁺ is detected with high sensitivity in the positive ion mode, and [M−H]⁻ or [M+HCOO]⁻ is detected with high sensitivity in the negative ion mode. Alternatively, it is also acceptable that a volatile acid such as formic acid, acetic acid, or trifluoroacetic acid is added to the ionization promoter liquid c.

The mass analysis apparatus 30 is configured to include an ionization apparatus 31 and a mass separation detection apparatus 32. In the mass analysis apparatus 30, a mixed solution of the ionization promoter liquid c and the eluents a and b is introduced through the connector 22, thereby lipid components including ceramides are ionized, and a mass analysis of the ionized lipid components is carried out.

Ionization of the ceramide component A and the ceramide component B introduced into the mass analysis apparatus 30 is carried out in the ionization apparatus 31.

The ionization method in the ionization apparatus 31 can be selected as appropriate. Specific examples of the ionization method include ESI, an atmospheric pressure chemical ionization (APCI) method, an atmospheric pressure photoionization method, a fast atom bombardment method, and a matrix-assisted laser desorption ionization method. Among these, from the viewpoint of detection sensitivity, an ESI method or an APCI method is preferred.

The mass separation detection apparatus 32 separates the ions produced in the ionization apparatus 31 at every m/z value and detects the separated ions. Regarding the mass separation detection apparatus 32, a mass spectrometer such as a quadrupole (Q) type mass spectrometer, an ion trap (IT) type mass spectrometer, or a time-of-flight (TOF) type mass spectrometer; a hybrid type mass spectrometer such as a Q-TOF type mass spectrometer or an IT-TOF type mass spectrometer; or a tandem mass spectrometer (MS/MS) such as a triple quadrupole type mass spectrometer can be used. Among these, a quadrupole (Q) type mass spectrometer is preferred.

According to the present invention, a commercially available liquid chromatograph-mass analysis apparatus in which the liquid chromatograph 10 and the mass analysis apparatus 30 are integrated may also be used.

The arithmetic unit 40 has an arithmetic means for triaxially expanding the retention time in the liquid chromatograph 10, and the m/z value and the ionic strength detected in the mass analysis apparatus 30, and thereby forming a multi-stage mass chromatogram.

Although not shown in the diagram, it is preferable that the arithmetic unit 40 can access to a database correlating the retention time and the m/z value for each and every molecular species in connection with the ceramides respectively corresponding to the ceramide component A and the ceramide component B. Furthermore, it is preferable that the arithmetic unit 40 has a comparison arithmetic means for using the multi-stage mass chromatogram formed by the arithmetic means as input data, retrieving the database based on the retention times of peaks included in the inputted multi-stage mass chromatogram and the m/z values, and specifying the ceramide molecular species corresponding to various peaks. Furthermore, it is preferable that the arithmetic unit 40 has a display means for outputting and displaying the multi-stage mass chromatogram formed by the arithmetic means, and/or the ceramide molecular species corresponding to the various peaks specified by the comparison arithmetic means, in a desired format.

The arithmetic unit 40 measures the content of ceramide component A and the content of ceramide component B from the multi-stage mass chromatogram formed by the arithmetic means. Then, the arithmetic unit 40 calculates the ratio of the quantitatively determined content of ceramide component A to the quantitatively determined content of ceramide component B.

The arithmetic unit 40 has an arithmetic means for evaluating the health of the skin of a test subject who is an object of evaluation, based on the information on the calculated ratio of the content of ceramide component A to the content of ceramide component B.

It is preferable that the arithmetic unit 40 stores a database associating the information on the ratio of the content of ceramide component A to the content of ceramide component B, both of which are included in a lipid sample prepared from a collected sample of the stratum corneum, with the health condition of the skin. Thus, from the calculated ratio of the content of ceramide component A to the content of ceramide component B, the health of the skin of the test subject who is an object of evaluation is evaluated based on the association function stored in the database described above.

According to the present invention, the health of the skin of a test subject can be evaluated from the calculated ratio of the content of ceramide component A to the content of ceramide component B of the test subject, by using the value distribution of the ratio of the content of ceramide component A to the content of ceramide component B, the value distribution having been produced according to the presence or absence of the onset of a skin disease, the progress of the skin disease, and the like. For example, in a case in which the NP component is selected as the ceramide component A and the NS component is selected as the ceramide component B, a value distribution of the ratio of the NP content to the NS content (hereinafter, also referred to as “NP/NS ratio”) produced by age of test subjects in regard to the health of the skin to be evaluated is used, thereby a deviation value of the NP/NS ratio of a test subject with respect to the average value obtained by age, to which the test subject belongs, and thus the good or bad of the health condition of the skin can be indicated. Alternatively, a reference value suitable for evaluating the health condition of the skin is determined from a graph obtained by plotting the health condition of the skin and the NP/NS ratio. Then, the health of the skin can be evaluated from a comparison between the reference value and the NP/NS ratio of a test subject. Furthermore, the health condition of the skin thus evaluated can also be visualized from the NP content and NS content thus calculated, and the ratio of the NP content to the NS content. For example, the NP content and the NS content thus calculated can be presented by the area of the chromatographic chart, and the health condition of the skin can be visually presented by the size of the area.

A ceramide molecule is a compound having a structure in which a sphingoid base and a fatty acid are bonded by an amide bond. A large number of ceramide classes such as NP and NS exist depending on the types of the sphingoid base and the fatty acid constituting a ceramide molecule (specifically, the presence or absence of a substituent, the number and positions of unsaturated bonds, and the like). Also, a large number of ceramide molecules having different numbers of carbon atoms of the sphingoid base and the fatty acid exist in the same ceramide class.

Among the compounds of ceramide component A described above, the term “NP” according to the present specification refers to a ceramide having a structure in which a phytosphingosine and a non-hydroxy fatty acid are bonded by an amide bond.

Further, the term “NH” according to the present specification refers to a ceramide having a structure in which a 6-hydroxysphingosine and a non-hydroxy fatty acid are bonded by an amide bond.

Further, the term “EOH” according to the present specification refers to a ceramide having a structure in which a 6-hydroxysphingosine and an esterified w-hydroxy fatty acid are bonded by an amide bond.

Further, the term “EOP” according to the present specification refers to a ceramide having a structure in which a phytosphingosine and an esterified w-hydroxy fatty acid are bonded by an amide bond.

Here, exemplary chemical structures of the NP component, the NH component, the EOH component, and the EOP component, all of which are included in the ceramide component A, are shown below. However, the present invention is not intended to be limited to these.

### Example of NP

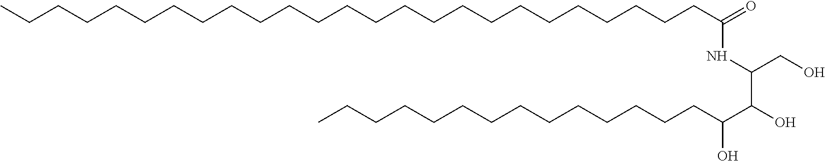 [SMILES: CCCCCCCCCCCCCCCCCCCCCCCCCC(=O)NC(CO)C(O)C(O)CCCCCCCCCCCCCC]

### Example of NH

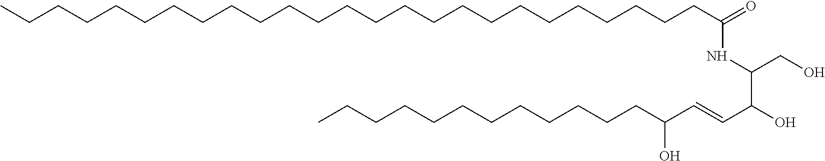 [SMILES: CCCCCCCCCCCCCCCCCCCCCCCCCC(=O)NC(CO)C(O)/C=C/C(O)CCCCCCCCCCCC]

### Example of EOH

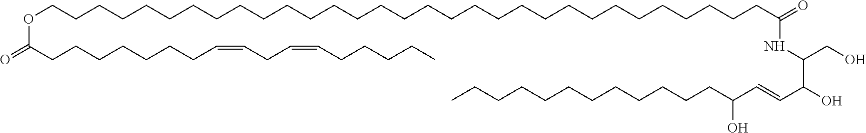 [SMILES: CCCCC/C=C\C/C=C\CCCCCCCC(=O)OCCCCCCCCCCCCCCCCCCCCCCCCCCCCCCCC(=O)NC(CO)C(O)/C=C/C(O)CCCCCCCCCCCC]

### Example of EOP

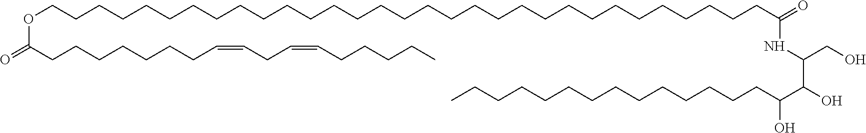 [SMILES: CCCCC/C=C\C/C=C\CCCCCCCC(=O)OCCCCCCCCCCCCCCCCCCCCCCCCCCCCCCCC(=O)NC(CO)C(O)C(O)CCCCCCCCCCCCCC]

The terms “phytosphingosine”, “sphingosine”, and “6-hydroxysphingosine” usually refer to amino alcohols having a structure with 18 carbon atoms. However, the “phytosphingosine”, “sphingosine”, and “6-hydroxysphingosine” in the present specification are regarded as generic names including amino alcohols having a structure with a number of carbon atoms of any number other than 18.

According to the present invention, there are no particular limitations on the number of carbon atoms of the phytosphingosine that constitutes the NP, and the number of carbon atoms is preferably 8 or higher, more preferably 16 or higher, preferably 44 or lower, and more preferably 36 or lower. Moreover, there are no particular limitations on the number of carbon atoms of the non-hydroxy fatty acid that constitutes the NP, and the number of carbon atoms is preferably 8 or higher, more preferably 16 or higher, preferably 44 or lower, and more preferably 36 or lower. Specific examples of the NP include N-hexadecanoyl-phytosphingosine, N-octadecanoyl-phytosphingosine, and N-tetracosanoyl-phytosphingosine.

According to the present invention, there are no particular limitations on the number of carbon atoms of the 6-hydroxysphingosine that constitutes the NH, and the number of carbon atoms is preferably 8 or higher, more preferably 16 or higher, preferably 44 or lower, and more preferably 36 or lower. Moreover, there are no particular limitations on the number of carbon atoms of the non-hydroxy fatty acid that constitutes the NH, and the number of carbon atoms is preferably 8 or higher, more preferably 16 or higher, preferably 44 or lower, and more preferably 36 or lower. Specific examples of the NH include N-hexadecanoyl-6-hydroxysphingosine, N-octadecanoyl-6-hydroxysphingosine, and N-tetracosanoyl-6-hydroxysphingosine.

According to the present invention, there are no particular limitations on the number of carbon atoms of the 6-hydroxysphingosine that constitutes the EOH, and the number of carbon atoms is preferably 8 or higher, more preferably 16 or higher, preferably 44 or lower, and more preferably 36 or lower. Moreover, there are no particular limitations on the number of carbon atoms of the esterified w-hydroxy fatty acid that constitutes the EOH, and the number of carbon atoms is preferably 30 or higher, more preferably 40 or higher, preferably 70 or lower, and more preferably 60 or lower. Specific examples of the EOH include N-(28-((linoleoyl)oxy)octacosanoyl)-6-hydroxysphingosine, N-(30-((linoleoyl)oxy)triacontanoyl)-6-hydroxysphingosine, and N-(32-((linoleoyl)oxy)dotriacontanoyl)-6-hydroxysphingosine.

According to the present invention, there are no particular limitations on the number of carbon atoms of the phytosphingosine that constitutes the EOP, and the number of carbon atoms is preferably 8 or higher, more preferably 16 or higher, preferably 44 or lower, and more preferably 36 or lower. Moreover, there are no particular limitations on the number of carbon atoms of the esterified w-hydroxy fatty acid that constitutes the EOP, and the number of carbon atoms is preferably 30 or higher, more preferably 40 or higher, preferably 70 or lower, and more preferably 60 or lower. Specific examples of the EOP include N-(28-((linoleoyl)oxy)octacosanoyl)-phytosphingosine, N-(30-((linoleoyl)oxy)triacontanoyl)-phytosphingosine, and N-(32-((linoleoyl)oxy)dotriacontanoyl)-phytosphingosine.

Among the compounds of ceramide component B described above, the term “NS” according to the present specification refers to a ceramide having a structure in which a sphingosine and a non-hydroxy fatty acid are bonded by an amide bond.

Further, the term “AS” according to the present specification refers to a ceramide having a structure in which a sphingosine and an α-hydroxy fatty acid are bonded by an amide bond.

Here, exemplary chemical structures of the NS component and the AS component, all of which are included in the ceramide component B, are shown below. However, the present invention is not intended to be limited to these.

### Example of NS

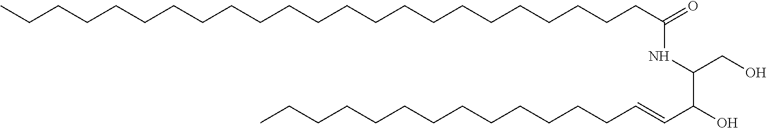 [SMILES: CCCCCCCCCCCCC/C=C/C(O)C(CO)NC(=O)CCCCCCCCCCCCCCCCCCCCCCC]

### Example of AS

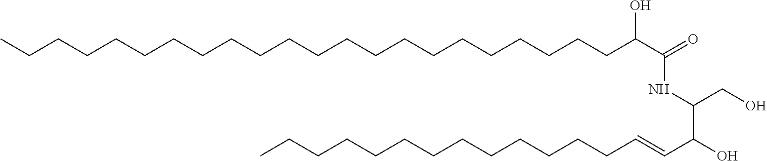 [SMILES: CCCCCCCCCCCCC/C=C/C(O)C(CO)NC(=O)C(O)CCCCCCCCCCCCCCCCCCCCCC]

According to the present invention, there are no particular limitations on the number of carbon atoms of the sphingosine that constitutes the NS, and the number of carbon atoms is preferably 8 or higher, more preferably 16 or higher, preferably 44 or lower, and more preferably 36 or lower. Moreover, there are no particular limitations on the number of carbon atoms of the non-hydroxy fatty acid that constitutes the NS, and the number of carbon atoms is preferably 8 or higher, more preferably 16 or higher, preferably 44 or lower, and more preferably 36 or lower. Specific examples of the NS include N-hexadecanoyl-sphingosine, N-octadecanoyl-sphingosine, and N-tetracosanoyl-sphingosine.

According to the present invention, there are no particular limitations on the number of carbon atoms of the sphingosine that constitutes the AS, and the number of carbon atoms is preferably 8 or higher, more preferably 16 or higher, preferably 44 or lower, and more preferably 36 or lower. Moreover, there are no particular limitations on the number of carbon atoms of the α-hydroxy fatty acid that constitutes the AS, and the number of carbon atoms is preferably 8 or higher, more preferably 16 or higher, preferably 44 or lower, and more preferably 36 or lower. Specific examples of the AS include α-hydroxyhexadecanoyl-sphingosine, α-hydroxyoctadecanoyl-sphingosine, and α-hydroxytetracosanoyl-sphingosine.

As shown in the following Examples, the ratio of the content of ceramide component A to the content of ceramide component B (specifically, the ratio of the NP content to the NS content, the ratio of the NH content to the NS content, the ratio of the EOH content to the NS content, the ratio of the EOP content to the NS content, the ratio of the NP content to the AS content, the ratio of the NH content to the AS content, the ratio of the EOH content to the AS content, or the ratio of the EOP content to the AS content) and the health condition of the skin highly correlate with each other. Therefore, the health of the skin of a test subject can be evaluated from the ratio of the content of ceramide component A to the content of ceramide component B, both amounts being quantitatively determined by the method described above.

The “evaluation of the health of the skin” according to the present specification means that an evaluation is made on whether the skin is in a healthy condition, and specifically that the skin condition for skin disease is evaluated, or the skin quality is evaluated.

Here, the phrase “skin condition for skin disease is evaluated” means that the skin condition related to skin disease, such as the presence or absence of the onset of a skin disease, the possibility of the onset of a skin disease, the prophylactic state of a skin disease, the progress of a skin disease, the presence or absence of a tendency (predisposition) toward a skin disease, the status of healing of a skin disease, and therapeutic effects for a skin disease, is evaluated.

The “skin disease” according to the present invention may be a skin disease in which inflammatory symptoms such as dermatitis, specifically symptoms such as pruritus, erythema, desquamation, scale, serous papule, and blisters are observed. Regarding the causes for skin disease, the disease may develop due to an external factor such as an irritant or an allergen, and the disease may develop due to an internal factor such as atopic predisposition. Furthermore, in many cases of skin diseases associated with inflammatory symptoms, the barrier function of the horny layer is impaired. Specific examples of the skin disease include contact dermatitis, atopic dermatitis, psoriasis, ichthyosis, hand eczema, asteatotic dermatitis, pityriasis alba, and lichen simplex. The present invention can be suitably used for an evaluation of the skin condition in atopic dermatitis and psoriasis as skin disease.

Furthermore, the phrase “skin quality is evaluated” means that the condition of skin including the scalp, such as the external appearance of the skin (luster, fineness of skin texture, presence or absence of desquamation, extent of desquamation, or the like), a sensitive skin, a dry skin, an oily skin, a skin having inferior moisturizing capacity, a skin having an inferior barrier function, an acne-prone skin, a scaling-prone skin, and an erythema-prone skin, is evaluated. Specifically, according to the present invention, the phrase includes evaluation of the skin quality such as the skin barrier function (transepidermal water loss), the horny layer water content, the skin color or skin luster, the skin texture, and desquamation of the skin (presence or absence of desquamation, or extent thereof).

According to the present invention, an evaluation of the health of the skin is carried out based on the evaluation criteria that have been previously set from the association function between the ratio of the content of ceramide component A to the content of ceramide component B and the health of the skin. In the present invention, the skin condition of a test subject is evaluated, based on the evaluation criteria described above, from the ratio of the content of ceramide component A to the content of ceramide component B, which is obtained from the measurement results for a lipid sample prepared from a collected sample of the stratum corneum of the test subject.

The evaluation criteria can be set as follows. However, the evaluation criteria are not limited to the following.

The health of the skin to be evaluated is evaluated by means such as evaluation by visual inspection and instrumental analysis. Apart from these, the ratio of the content of ceramide component A to the content of ceramide component B in a lipid sample prepared from a collected sample of the stratum corneum by the method described above (hereinafter, also simply referred to as “content ratio”) is calculated. Then, reference values suitable for evaluating the health condition of the skin are determined based on the correlation between the evaluation results for the health of the skin and the content ratio, and evaluation criteria are set based on the reference values. The evaluation criteria can be set by race, by gender, or by age of the test subject, in accordance with the test subject as an object of evaluation of the health of the skin, or with the purpose of the evaluation.

For example, in the case of evaluating the skin condition for skin disease as the health of the skin, a healthy group consisting of test subjects who are considered to have sound health of the skin, and a non-healthy group consisting of test subjects who are not considered to have good health (hereinafter, also called “trouble group”) are produced based on the evaluation results for the health of the skin. It is also acceptable to produce three or more groups depending on the skin condition with which the evaluation is carried out. Groups are similarly produced also for the case of evaluating the skin quality as the health of the skin.

The value ranges of the content ratio that characterize the respective groups are determined based on the statistical analysis results of the content ratio of the test subjects who belong to the respective groups. These value ranges are each determined by setting a certain range to include an upper limit and a lower limit centered about the average value of each of the groups. Here, regarding the “certain range”, a statistical value such as the standard deviation (SD), a ½ SD value, a ⅓ SD value, and the like may be used, or any arbitrary value that has been set in advance may also be used. It is preferable that the value range of the ratio that characterizes each of the groups is set so as not to include the average values of other groups within the set value range. Then, the upper limit or the lower limit of the value range that characterizes each of the groups is employed as the reference value used for the evaluation criteria.

In the method of setting the evaluation criteria using reference values, for example, when the average value of the content ratio of the healthy group is higher than the average value of the content ratio of the non-healthy group, the lower limit of the value range of the content ratio of the healthy group or the upper limit of the value range of the content ratio of the non-healthy group is employed as the reference value, and the evaluation criteria is set such that the case in which the calculated content ratio is greater than or equal to the reference value (or the case in which the calculated content ratio is greater than the reference value) is evaluated as “healthy”. Whereas, the case in which the calculated content ratio is less than the reference value (or the case in which the calculated content ratio is less than or equal to the reference value) is evaluated as “having a possibility of being not healthy (having trouble)”.

On the other hand, when the average value of the content ratio of the healthy group is lower than the average value of the content ratio of the non-healthy group, the upper limit of the value range of the content ratio of the healthy group or the lower limit of the value range of the content ratio of the non-healthy group is employed as the reference value, and the evaluation criteria can be set such that the case in which the calculated content ratio is less than the reference value (or the case in which the calculated content ratio is less than or equal to the reference value) is evaluated as “healthy”. Whereas, the case in which the calculated content ratio is greater than or equal to the reference value (or the case in which the calculated content ratio is greater than the reference value) is evaluated as “having a possibility of being not healthy (having trouble)”. The evaluation criteria may also be set by using a plurality of reference values in combination.

Furthermore, in a case in which the skin quality such as the skin barrier function, the horny layer water content, the skin luster or skin color, and the skin characteristics are evaluated as the health of the skin, a reference value used for the evaluation criteria may be determined from a graph obtained by plotting the evaluation results for the health of the skin and the content ratio, and thus the evaluation criteria may be set. Specifically, with regard to the plotted graph, test subjects are divided into a healthy group and a non-healthy group (trouble group) or more groups, based on the indicators of the health of the skin (TEWL value, Capacitance, L* value, a* value, score value, and the like), reference values are determined from the distribution state of the plot of the respective groups, and the evaluation criteria on whether the skin quality or the like is good can be set based on the reference values. The evaluation criteria may also be set by using a plurality of reference values in combination.

In regard to a method of evaluating the skin condition for atopic dermatitis and psoriasis in the arm as a specific embodiment of the method of evaluating the health of the skin of the present invention, the evaluation criteria of using specific reference values will be explained. However, the present invention is not intended to be limited to these.

The “ratio of the content of ceramide component A to the content of ceramide component B” according to the present specification can be specifically indicated, such as (content of ceramide component A):(content of ceramide component B), (content of ceramide component B):(content of ceramide component A), (content of ceramide component A)/(content of ceramide component B), or (content of ceramide component B)/(content of ceramide component A). Among such expression formats, in the following description, the “ratio of the content of ceramide component A to the content of ceramide component B” is described in the form of “(content of ceramide component A)/(content of ceramide component B)”. However, in the present invention, the “ratio of the content of ceramide component A to the content of ceramide component B” may also be expressed in some other form.

The value ranges described below are all described on a mass basis.

Evaluation criteria that use specific reference values according to the first embodiment of the present invention will be explained.

It is desirable that: when NP/NS ratio of a stratum corneum-derived lipid sample collected from a non-rash part or a healthy part is 2.7 or higher, the test subject can be evaluated to be healthy; when the NP/NS ratio is less than 2.1, the test subject can be evaluated to have a possibility of atopic dermatitis; and when the NP/NS ratio is less than 1.6, the test subject can be evaluated to have a possibility of psoriasis. Here, the (average value of healthy group−SD) and the (average value of rash part of non-healthy group (skin disease group)+SD) were used in combination as reference values.

Next, evaluation criteria that use specific reference values according to the second embodiment of the present invention will be explained.

It is desirable that: when NH/NS ratio of a stratum corneum-derived lipid sample collected from a non-rash part or a healthy part is 3.2 or higher, the test subject can be evaluated to be healthy; when the NH/NS ratio is less than 2.3, the test subject can be evaluated to have a possibility of atopic dermatitis; and when the NH/NS ratio is less than 1.5, the test subject can be evaluated to have a possibility of psoriasis.

It is desirable that: when EOH/NS ratio of a stratum corneum-derived lipid sample collected from a non-rash part or a healthy part is 0.3 or higher, the test subject can be evaluated to be healthy; when the EOH/NS ratio is less than 0.3, the test subject can be evaluated to have a possibility of atopic dermatitis; and when the EOH/NS ratio is less than 0.2, the test subject can be evaluated to have a possibility of psoriasis.

It is desirable that: when EOP/NS ratio of a stratum corneum-derived lipid sample collected from a non-rash part or a healthy part is 0.1 or higher, the test subject can be evaluated to be healthy; and when the EOP/NS ratio is less than 0.1, the test subject can be evaluated to have a possibility of atopic dermatitis or psoriasis.

It is desirable that: when NP/AS ratio of a stratum corneum-derived lipid sample collected from a non-rash part or a healthy part is 4.5 or higher, the test subject can be evaluated to be healthy; when the NP/AS ratio is less than 2.6, the test subject can be evaluated to have a possibility of atopic dermatitis; and when the NP/AS ratio is less than 2.1, the test subject can be evaluated to have a possibility of psoriasis.

It is desirable that: when NH/AS ratio of a stratum corneum-derived lipid sample collected from a non-rash part or a healthy part is 4.9 or higher, the test subject can be evaluated to be healthy; when the NH/AS ratio is less than 2.8, the test subject can be evaluated to have a possibility of atopic dermatitis; and when the NH/AS ratio is less than 2.0, the test subject can be evaluated to have a possibility of psoriasis.

It is desirable that: when EOH/AS ratio of a stratum corneum-derived lipid sample collected from a non-rash part or a healthy part is 0.5 or higher, the test subject can be evaluated to be healthy; when the EOH/AS ratio is less than 0.3, the test subject can be evaluated to have a possibility of atopic dermatitis; and when the EOH/AS ratio is less than 0.2, the test subject can be evaluated to have a possibility of psoriasis.

It is desirable that: when EOP/AS ratio of a stratum corneum-derived lipid sample collected from a non-rash part or a healthy part is 0.2 or higher, the test subject can be evaluated to be healthy; and when the EOP/AS ratio is less than 0.1, the test subject can be evaluated to have a possibility of atopic dermatitis or psoriasis.

Among the specific examples of the reference values described above, in regard to the NH/NS ratio, the EOP/NS ratio, the NP/AS ratio, the NH/AS ratio, the EOH/AS ratio, and the EOP/AS ratio, the (average value of healthy group −SD) and the (average value of rash part of non-healthy group (skin disease group)+SD) were used in combination as reference values. In regard to the EOH/NS ratio, the (average value of rash part of non-healthy group (skin disease group)+SD) was used as a reference value.

In regard to the skin quality such as the skin barrier function, the horny layer water content, the desquamation, the skin texture, and the skin color or skin luster among the factors of the health of the skin, the evaluation criteria based on the ratio of the content of ceramide component A to the content of ceramide component B will be explained by way of specific examples. However, the present invention is not intended to be limited to these.

The NH/NS ratio of a lipid sample collected from the buccal region of a test subject having a transepidermal water loss (TEWL) of greater than 20 was less than 1.5 in most cases. Here, generally, when the TEWL is 20 or less, the skin barrier function is evaluated to be normal, or the skin barrier function is evaluated to be higher than or equal to the average (see Yamashita Y., et al., Skin Pharmacol. Physiol., 2012, vol. 25, p. 78-85; and Gae W. N., et al., Journal of Cosmetics, Dermatological Sciences and Applications, 2014, vol. 4, p. 44-52). Therefore, the reference value for the NH/NS ratio in connection to the skin barrier function can be determined to be 1.5, and evaluation can be carried out such that when the NH/NS ratio of a stratum corneum-derived lipid sample is 1.5 or higher, it is considered that the “skin barrier function is normal” or the “skin barrier function is higher than or equal to the average”. To the contrary, when the NH/NS ratio is less than 1.5, it considered that “there is a possibility that the skin barrier function may be abnormal”.

In regard to the skin barrier function, reference values can also be determined as appropriate for ratios of the content of ceramide component A to the content of ceramide component B, the ratios being other than the NH/NS ratio, and evaluation can be carried out similarly.

The EOH/NS ratio of a lipid sample collected from the buccal region of a test subject having a horny layer water content (Capacitance) of higher than 60 was approximately 0.15 or greater. Therefore, the reference value for the EOH/NS ratio related to the horny layer water content can be determined to be 0.15, and evaluation can be carried out such that when the EOH/NS ratio of a stratum corneum-derived lipid sample is 0.15 or greater, it is considered that the “horny layer water content is large” or the “horny layer water content is higher than or equal to the average”. To the contrary, when the EOH/NS ratio is less than 0.15, it is considered that “there is a possibility that the horny layer water content may be low”.

In regard to the horny layer water content, reference values can also be determined as appropriate for ratios of the content of ceramide component A to the content of ceramide component B, the ratios being other than the EOH/NS ratio, and evaluation can be carried out similarly.

The EOP/NS ratio of a lipid sample collected from the buccal region of a test subject who showed desquamation from the buccal region was less than 0.05. Therefore, the reference value for the EOP/NS ratio related to desquamation can be determined as 0.05, and evaluation can be carried out such that when the EOP/NS ratio of a stratum corneum-derived lipid sample is 0.05 or higher, it is considered that “desquamation is not at all observed” or “desquamation is slightly observed”, while when the EOP/NS ratio is less than 0.05, it is considered that “there is a possibility that desquamation may be observed”.

In regard to desquamation, reference values can also be determined as appropriate for ratios of the content of ceramide component A to the content of ceramide component B, the ratios being other than the EOP/NS ratio, and evaluation can be carried out similarly.

The skin texture score of a test subject having a NH/NS ratio of 1.6 or higher for a lipid sample collected from the buccal region was 2.5 or greater in most cases, and the skin texture was well-ordered. Therefore, the reference value for the NH/NS ratio related to the skin texture can be determined to be 1.6, and evaluation can be carried out such that when the NH/NS ratio of a stratum corneum-derived lipid sample is 1.6 or higher, it is considered that the “skin texture is well-ordered” or the “skin texture is fine”, while when the NH/NS ratio is less than 1.6, it is considered that “there is a possibility that the skin texture may be disordered”.

In regard to the skin texture, reference values can also be determined as appropriate for ratios of the content of ceramide component A to the content of ceramide component B, the ratios being other than the NH/NS ratio, and evaluation can be carried out similarly.

The L* value of a test subject having a NP/AS ratio of 2.0 or higher for a lipid sample collected from the buccal region was approximately 65 or greater. Here, generally, when the L* value is 65 or greater, the skin is evaluated such that the skin color is bright, or the skin has a healthy skin color (see Caisey L., et al., International Journal of Cosmetic Science, 2006, vol. 28, p. 427-437, and the like). Therefore, the reference value for the NP/AS ratio related to the L* value can be determined to be 2.0, and evaluation can be carried out such that when the NP/AS ratio of a stratum corneum-derived lipid sample is 2.0 or greater, it is considered that the “skin color is bright” or the “skin has a healthy skin color”, while when the NP/AS ratio is less than 2.0, it is considered that “there is a possibility that the skin color may be dark” or “there is a possibility that the skin color may not be healthy”.

In regard to the L* value, reference values can also be determined as appropriate for ratios of the content of ceramide component A to the content of ceramide component B, the ratios being other than the NP/AS ratio, and evaluation can be carried out similarly.

The NP/AS ratio of a lipid sample collected from the buccal region of a test subject having an a* value of 14 or higher was approximately less than 2.0. Therefore, the reference value for the NP/AS ratio related to the a* value can be determined to be 2.0, and evaluation can be carried out such that when the NP/AS ratio of a stratum corneum-derived lipid sample is 2.0 or greater, it is considered that the “skin is less reddish tinged”, while when the NP/AS ratio is less than 2.0, it is considered that “there is a possibility that the skin may be more reddish tinged”.

In regard to the a* value, reference values can also be determined as appropriate for ratios of the content of ceramide component A to the content of ceramide component B, the ratios being other than the NP/AS ratio, and evaluation can be carried out similarly.

As disclosed in the Examples described below, the ratio of the content of ceramide component A to the content of ceramide component B shows high correlativity with the health of the skin, such as skin disease or skin quality. Therefore, the ratio of the content of ceramide component A to the content of ceramide component B in the stratum corneum serves as an index for evaluating the health of the skin, and the health of the skin can be evaluated conveniently and accurately by measuring this ratio. Furthermore, according to the method of evaluating the health of the skin of the present invention, in a test for the application of an external medicine for skin, a test for the intake of a certain functional food, pharmaceutical product, quasi-drug, or the like, the effectiveness for the prevention or amelioration of skin disease or an improvement of the skin quality provided by such a test substance can be judged by measuring the amount of change in the ratio of the content of ceramide component A to the content of ceramide component B in the stratum corneum, caused by application or intake of the test substance.

As explained above, in the method of evaluating the health of the skin of the present invention, an evaluation of the health of the skin of a test subject is carried out by using the ratio of the content of ceramide component A to the content of ceramide component B as an index. Then, by using the ratio of the content of ceramide component A to the content of ceramide component B as an index, the health of the skin can be accurately evaluated from various viewpoints, such as the skin condition such as the presence or absence of the onset of skin diseases such as atopic dermatitis and psoriasis, the possibility of the onset of the skin disease, the prophylactic state of the skin disease, the progress of the skin disease, the presence or absence of a tendency (predisposition) toward the skin disease, the status of healing of the skin disease, and therapeutic effects for the skin disease; and the skin quality such as the skin barrier function, the horny layer water content, the skin color or skin luster, the skin texture, and the presence or absence of desquamation.

Furthermore, in the method of evaluating the health of the skin of the present invention, the ratio of the content of ceramide component A to the content of ceramide component B is used as an index. Therefore, in the method of evaluating the health of the skin of the present invention, when the amounts of any desired two kinds of ceramide classes (ceramide component A and ceramide component B) are quantitatively determined, the ratio can be calculated. Therefore, an analysis of the molecular species of the ceramides included in the lipid sample, or the calculation of the total amount of ceramides and the proportions (composition ratio) occupied by the respective ceramide classes with respect to the total amount of ceramides, is not needed. Furthermore, calculation of the area of the stratum corneum that has been detached in order to standardize the ceramide components, the weight of the stratum corneum, the amount of proteins, or the quantitative value obtained by using the number of cells or the like is also not necessary. Therefore, according to the method of evaluating the health of the skin of the present invention, evaluation of the health of the skin can be carried out more conveniently, compared to conventional methods.

By utilizing the method of evaluating the health of the skin of the present invention, or by using an apparatus for evaluating the health of the skin, a prophylactic or ameliorating agent for skin disease, or a skin quality improving agent can be screened. Specifically, an external agent for skin, a cosmetic product, a pharmaceutical product, a quasi-drug, a food or the like, all of which contains a candidate substance for a prophylactic or ameliorating agent for skin disease or a skin quality improving agent, is applied on the skin or a test subject or is orally administered, any change in the health of the skin before and after the application or administration of the external agent for skin, cosmetic product, pharmaceutical product, quasi-drug, food, or the like is checked by carrying out the method of the present invention or by using an apparatus for evaluating the health of the skin, and a substance that provides a prophylactic or ameliorating effect for skin disease, or a substance that provides a skin quality improving effect can be selected as a prophylactic or ameliorating agent for skin disease or a skin quality improving agent.

The term “prophylaxis” according to the present invention means that prevention or delay of the onset of disease or symptom in an individual, or reduction of a risk of the onset of disease or symptom in an individual. Specifically, the term implies that when the average value of the content ratio of a healthy group is higher than the average value of the content ratio of a non-healthy group, at least one content ratio among the content ratios described above, and preferably all of the content ratios, are maintained in a state of being higher than the reference value described above. On the other hand, the term implies that when the average value of the content ratio of the healthy group is lower than the average value of the content ratio of the non-healthy group, at least one content ratio among the content ratios described above, and preferably all of the content ratios, are maintained in a state of being lower than the reference value described above.

For example, in regard to atopic dermatitis, the term prophylaxis preferably implies that at least one value range, and preferably all of the value ranges, selected from a NP/NS ratio of 2.1 or greater, a NH/NS ratio of 2.3 or greater, an EOH/NS ratio of 0.3 or greater, an EOP/NS ratio of 0.1 or greater, a NP/AS ratio of 2.6 or greater, a NH/AS ratio of 2.8 or greater, an EOH/AS ratio of 0.3 or greater, and an EOP/AS ratio of 0.1 or greater, all obtained from a stratum corneum-derived lipid sample collected from a non-rash part or a healthy part is maintained. Further, in regard to psoriasis, the term prophylaxis preferably implies that at least one value range, and preferably all of the value ranges, selected from a NP/NS ratio of 1.6 or greater, a NH/NS ratio of 1.5 or greater, an EOH/NS ratio of 0.2 or greater, an EOP/NS ratio of 0.1 or greater, a NP/AS ratio of 2.1 or greater, a NH/AS ratio of 2.0 or greater, an EOH/AS ratio of 0.2, and an EOP/AS ratio of 0.1 or greater, all obtained from a stratum corneum-derived lipid sample collected from a non-rash part or a healthy part is maintained.

Further, the term “amelioration” as used herein means an improvement or alleviation of disease, symptom or condition, prevention or delay of aggravation of disease, symptom or condition, or reverse, prevention or delay of the progress of disease, symptom or condition. Specifically, the term amelioration implies that when the average value of the content ratio of the healthy group is higher than the average value of the content ratio of the non-healthy group, at least one content ratio among the content ratios described above, and preferably all of the content ratios, are in a state of being greater than the reference values described above. On the other hand, the term implies that when the average value of the content ratio of the healthy group is smaller than the average value of the content ratio of the non-healthy group, at least one content ratio among the content ratios described above, and preferably all of the content ratios, are in a state of being smaller than the reference values described above.

For example, in regard to atopic dermatitis, the term amelioration preferably implies that at least one value range, and preferably all of the value ranges, selected from a NP/NS ratio of 2.1 or greater, a NH/NS ratio of 2.3 or greater, an EOH/NS ratio of 0.3 or greater, an EOP/NS ratio of 0.1 or greater, a NP/AS ratio of 2.6 or greater, a NH/AS ratio of 2.8 or greater, an EOH/AS ratio of 0.3 or greater, and an EOP/AS ratio of 0.1 or greater, all obtained from a stratum corneum-derived lipid sample collected from a non-rash part, is attained. Further, in regard to psoriasis, the term amelioration preferably implies that at least one value range, and preferably all of the value ranges, selected from a NP/NS ratio of 1.6 or greater, a NH/NS ratio of 1.5 or greater, an EOH/NS ratio of 0.2 or greater, an EOP/NS ratio of 0.1 or greater, a NP/AS ratio of 2.1 or greater, a NH/AS ratio of 2.0 or greater, an EOH/AS ratio of 0.2 or greater, and an EOP/AS ratio of 0.1 or greater, all obtained from a stratum corneum-derived lipid sample collected from a non-rash part, is attained.

In regard to the embodiments described above, the present invention further discloses the following method of evaluating the health of the skin, apparatus for evaluating the health of the skin, and method of screening a prophylactic or ameliorating agent for skin disease.

<1> A method of evaluating the health of skin, containing the steps of:

quantitatively determining respective contents of a NP component and a NS component included in a lipid sample prepared from a collected sample of a stratum corneum of a test subject;

calculating the content ratio of the quantitatively determined content of the NP component to the quantitatively determined content of the NS component; and

evaluating the skin condition for skin disease (preferably atopic dermatitis or psoriasis) of the test subject from the calculated content ratio.

<2> A method of evaluating the health of skin, containing the steps of:

quantitatively determining respective contents of one kind of ceramide component A and one kind of ceramide component B included in a lipid sample prepared from a collected sample of a stratum corneum of a test subject;

calculating the content ratio of the quantitatively determined content of the ceramide component A to the quantitatively determined content of the ceramide component B; and

evaluating the health of the skin of the test subject from the calculated content ratio;

wherein the ceramide component A is selected from the group consisting of a NP component, a NH component, an EOH component, and an EOP component; and the ceramide component B is selected from the group consisting of a NS component and an AS component; provided that the case of selecting the NP component as the ceramide component A and the NS component as the ceramide component B is excluded.
<3> A method for evaluating the skin condition for a skin disease (preferably atopic dermatitis or psoriasis) as an evaluation of the health of skin, containing the steps of:

quantitatively determining respective contents of a NP component and a NS component included in a lipid sample prepared from a collected sample of a stratum corneum of a test subject; and

calculating the content ratio of the quantitatively determined content of the NP component to the quantitatively determined content of the NS component.

<4> A method for evaluating the health of skin, containing the steps of:

quantitatively determining respective contents of one kind of ceramide component A and one kind of ceramide component B included in a lipid sample prepared from a collected sample of a stratum corneum of a test subject; and

calculating the content ratio of the quantitatively determined content of the ceramide component A to the quantitatively determined content of the ceramide component B;

wherein the ceramide component A is selected from the group consisting of a NP component, a NH component, an EOH component, and an EOP component; and

the ceramide component B is selected from the group consisting of a NS component and an AS component; provided that the case of selecting the NP component as the ceramide component A and the NS component as the ceramide component B is excluded.

<5> The method described in any one of the above items <1> to <4>, wherein the lipid sample is a lipid sample prepared from a collected sample of the stratum corneum from a non-rash part of a test subject or from a healthy part of a test subject who has not developed skin disease.
<6> The method described in any one of the above items <1> to <5>, wherein the health of the skin is evaluated, based on the association function between the information of the ratio of the content of one kind of ceramide component A selected from the group consisting the NP component, the NH component, the EOH component and the EOP component, to the content of one kind of ceramide component B selected from the group consisting of the NS component and the AS component, both ceramide components being included in the lipid sample prepared from a collected sample of a stratum corneum, and the health condition of the skin, from the ratio of the quantitatively determined content of the ceramide component A to the quantitatively determined content of the ceramide component B.

<7> The method described in any one of the above items <1> to <6>, wherein the health of the skin relates to a skin disease (preferably atopic dermatitis or psoriasis) or a skin quality (preferably at least one selected from the group consisting of the skin barrier function, the horny layer water content, the skin color or skin luster, the skin texture, and the desquamation of skin; and more preferably the skin barrier function, the horny layer water content, the skin color or skin luster, the skin texture, and the desquamation of skin).

<8> The method described in any one of the above items <1> to <7>, wherein the ceramide components are respectively quantitatively determined by an LC-MS method.
<9> The method described in the above item <8>, wherein in the LC-MS method, the ceramide components are respectively separated by liquid chromatography; the ceramide components thus separated are respectively ionized by any one of an ESI method, an APCI method, an atmospheric pressure photoionization method, a fast atom bombardment method, and a matrix-assisted laser desorption ionization method, and preferably by an ESI method; and the ceramide components thus ionized are respectively quantitatively determined with a mass separation detection apparatus.
<10> The method described in any one of the above items <1> to <9>, wherein the health of the skin of a human being or a mammal other than a human being is evaluated.

<11> The method described in any one of the above items <1> to <10>, wherein the stratum corneum is collected by a tape stripping method, and a lipid sample is prepared from the stratum corneum thus collected.

<12> The method described in the above item <11>, wherein the stratum corneum collected by a tape stripping method is immersed in methanol and ultrasonically treated, and thus a lipid sample is prepared.
<13> An apparatus for evaluating the health of skin, containing:

a quantitative determination means for quantitatively determining respective contents of a NP component and a NS component included in a lipid sample prepared from a collected sample of a stratum corneum; and

an arithmetic means for calculating the content ratio of the quantitatively determined content of the NP component to the quantitatively determined content of the NS component, and evaluating the skin condition for skin disease (preferably atopic dermatitis or psoriasis) of a test subject from the calculated content ratio.

<14> An apparatus for evaluating the health of skin, containing:

a quantitative determination means for quantitatively determining respective contents of one kind of ceramide component A and one kind of ceramide component B included in a lipid sample prepared from a collected sample of a stratum corneum; and

an arithmetic means for calculating the content ratio of the quantitatively determined content of the ceramide component A to the quantitatively determined content of the ceramide component B, and evaluating the health of the skin of a test subject from the calculated content ratio;

wherein the ceramide component A is selected from the group consisting of a NP component, a NH component, an EOH component, and an EOP component; and the ceramide component B is selected from the group consisting of a NS component and an AS component; provided that the case of selecting the NP component as the ceramide component A and the NS component as the ceramide component B is excluded.
<15> The apparatus described in the above item <13> or <14>, wherein the apparatus stores a database that associates the information of the ratio of the content of one kind of ceramide component A selected from the group consisting of the NP component, the NH component, the EOH component and the EOP component, to the content of one kind of ceramide component B selected from the group consisting of the NS component and the AS component, with the health condition of the skin; and
wherein the health of the skin is evaluated from the ratio of the content of ceramide component A to the content of ceramide component B calculated by the arithmetic means based on the association function of the database.
<16> The apparatus described in any one of the above items <13> to <15>, wherein the health of the skin relates to a skin disease (preferably atopic dermatitis or psoriasis) or a skin quality (preferably at least one selected from the group consisting of the skin barrier function, the horny layer water content, the skin color or skin luster, the skin texture, and the desquamation of skin; and more preferably the skin barrier function, the horny layer water content, the skin color or skin luster, the skin texture, and the desquamation of skin).
<17> The apparatus described in any one of the above items <13> to <16>, wherein the quantitative determination means quantitatively determines each of the ceramide components by an LC-MS method.
<18> The apparatus described in the above item <17>, wherein in the LC-MS method, the ceramide components are respectively separated by liquid chromatography; the ceramide components thus separated are respectively ionized by any one of an ESI method, an atmospheric pressure chemical ionization method, an atmospheric pressure photoionization method, a fast atom bombardment method, and a matrix-assisted laser desorption ionization method, and preferably by an ESI method; and the ceramide components thus ionized are respectively quantitatively determined.
<19> The method or apparatus according to any one of the above items <1> to <18>, wherein the content ratio is the ratio of the NP content to the NS content, the ratio of the NH content to the NS content, the ratio of the EOH content to the NS content, the ratio of the EOP content to the NS content, the ratio of the NP content to the AS content, the ratio of the NH content to the AS content, the ratio of the EOH content to the AS content, or the ratio of the EOP content to the AS content.
<20> The method or apparatus according to any one of the above items <1> to <19>,
wherein the number of carbon atoms of the phytosphingosine that constitutes the NP is 8 or higher, preferably 16 or higher, and the upper limit thereof is 44 or lower, preferably 36 or lower; and
wherein the number of carbon atoms of the non-hydroxy fatty acid that constitutes the NP is preferably 8 or higher, preferably 16 or higher, and the upper limit thereof is 44 or lower, preferably 36 or lower.
<21> The method or apparatus according to any one of the above items <1> to <19>,
wherein the number of carbon atoms of the 6-hydroxysphingosine that constitutes the NH is 8 or higher, preferably 16 or higher, and the upper limit thereof is 44 or lower, preferably 36 or lower; and
wherein the number of carbon atoms of the non-hydroxy fatty acid that constitutes the NH is preferably 8 or higher, preferably 16 or higher, and the upper limit thereof is 44 or lower, preferably 36 or lower.

<22> The method or apparatus according to any one of the above items <1> to <19>,

wherein the number of carbon atoms of the 6-hydroxysphingosine that constitutes the EOH is 8 or higher, preferably 16 or higher, and the upper limit thereof is 44 or lower, preferably 36 or lower; and
wherein the number of carbon atoms of the esterified w-hydroxy fatty acid that constitutes the EOH is preferably 30 or higher, preferably 40 or higher, and the upper limit thereof is 70 or lower, preferably 60 or lower.

<23> The method or apparatus according to any one of the above items <1> to <19>,

wherein the number of carbon atoms of the phytosphingosine that constitutes the EOP is 8 or higher, preferably 16 or higher, and the upper limit thereof is 44 or lower, preferably 36 or lower; and
wherein the number of carbon atoms of the esterified w-hydroxy fatty acid that constitutes the EOP is preferably 30 or higher, preferably 40 or higher, and the upper limit thereof is 70 or lower, preferably 60 or lower.
<24> The method or apparatus according to any one of the above items <1> to <19>,
wherein the number of carbon atoms of the sphingosine that constitutes the NS is 8 or higher, preferably 16 or higher, and the upper limit thereof is 44 or lower, preferably 36 or lower; and wherein the number of carbon atoms of the non-hydroxy fatty acid that constitutes the NS is preferably 8 or higher, preferably 16 or higher, and the upper limit thereof is 44 or lower, preferably 36 or lower.
<25> The method or apparatus according to any one of the above items <1> to <19>,
wherein the number of carbon atoms of the sphingosine that constitutes the AS is 8 or higher, preferably 16 or higher, and the upper limit thereof is 44 or lower, preferably 36 or lower; and
wherein the number of carbon atoms of the α-hydroxy fatty acid that constitutes the AS is preferably 8 or higher, preferably 16 or higher, and the upper limit thereof is 44 or lower, preferably 36 or lower.
<26> The method or apparatus described in any one of the above items <1> to <25>,
wherein in a case in which the average value of the ceramide content ratio of the healthy group is higher than the average value of the ceramide content ratio of the non-healthy group,

when the ceramide content ratio thus calculated is higher than the lower limit of the value range of the ceramide content ratio characterizing the healthy group or the upper limit of the value range of the ceramide content ratio characterizing the non-healthy group, the test subject is evaluated as “being healthy”, and

when the ceramide content ratio thus calculated is lower than or equal to the lower limit of the value range of the ceramide content ratio characterizing the healthy group or the upper limit of the value range of the ceramide content ratio characterizing the non-healthy group, the test subject is evaluated as “having a possibility of being non-healthy (having trouble)” or “having a high possibility of being non-healthy (having trouble)”.

<27> The method or apparatus described in any one of the above items <1> to <25>,
wherein in a case in which the average value of the ceramide content ratio of the healthy group is lower than the average value of the ceramide content ratio of the non-healthy group,

when the ceramide content ratio thus calculated is lower than the upper limit of the value range of the ceramide content ratio characterizing the healthy group or the lower limit of the value range of the ceramide content ratio characterizing the non-healthy group, the test subject is evaluated as “being healthy”, and

when the ceramide content ratio thus calculated is higher than or equal to the upper limit of the value range of the ceramide content ratio characterizing the healthy group or the value or the lower limit of the value range of the ceramide content ratio characterizing the non-healthy group, the test subject is evaluated as “having a possibility of being non-healthy (having trouble)” or “having a high possibility of being non-healthy (having trouble)”.

<28> The method or apparatus described in any one of the above items <1> to <27>, wherein the presence or absence of the onset of a skin disease, the possibility of the onset of a skin disease, the prophylactic state of a skin disease, the progress of a skin disease, the presence or absence of a tendency (predisposition) toward a skin disease, the status of healing of a skin disease, or therapeutic effects for a skin disease, is evaluated as the skin health.
<29> The method or apparatus described in the above item <28>, wherein the skin disease is atopic dermatitis or psoriasis.
<30> The method or apparatus described in any one of the above items <1> to <29>, wherein the evaluation is carried out for atopic dermatitis by using the ratio of the NP content to the NS content as an index.
<31> The method or apparatus described in any one of the above items <1> to <30>, wherein the evaluation is carried out for psoriasis by using the ratio of the NP content to the NS content as an index.
<32> The method or apparatus described in any one of the above items <1> to <29>, wherein the evaluation is carried out for atopic dermatitis by using the ratio of the NH content to the NS content as an index.
<33> The method or apparatus described in any one of the above items <1> to <28>, wherein the evaluation is carried out for psoriasis by using the ratio of the NH content to the NS content as an index.
<34> The method or apparatus described in any one of the above items <1> to <29>, wherein the evaluation is carried out for atopic dermatitis by using the ratio of the EOH content to the NS content as an index.
<35> The method or apparatus described in any one of the above items <1> to <28>, wherein the evaluation is carried out for psoriasis by using the ratio of the EOH content to the NS content as an index.
<36> The method or apparatus described in any one of the above items <1> to <29>, wherein the evaluation is carried out for atopic dermatitis by using the ratio of the EOP content to the NS content as an index.
<37> The method or apparatus described in any one of the above items <1> to <28>, wherein the evaluation is carried out for psoriasis by using the ratio of the EOP content to the NS content as an index.
<38> The method or apparatus described in any one of the above items <1> to <29>, wherein the evaluation is carried out for atopic dermatitis by using the ratio of the NP content to the AS content as an index.
<39> The method or apparatus described in any one of the above items <1> to <28>, wherein the evaluation is carried out for psoriasis by using the ratio of the NP content to the AS content as an index.
<40> The method or apparatus described in any one of the above items <1> to <29>, wherein the evaluation is carried out for atopic dermatitis by using the ratio of the NH content to the AS content as an index.
<41> The method or apparatus described in any one of the above items <1> to <29>, wherein the evaluation is carried out for psoriasis by using the ratio of the NH content to the AS content as an index.
<42> The method or apparatus described in any one of the above items <1> to <29>, wherein the evaluation is carried out for atopic dermatitis by using the ratio of the EOH content to the AS content as an index.
<43> The method or apparatus described in any one of the above items <1> to <30>, wherein the evaluation is carried out for psoriasis by using the ratio of the EOH content to the AS content as an index.
<44> The method or apparatus described in any one of the above items <1> to <29>, wherein the evaluation is carried out for atopic dermatitis by using the ratio of the EOP content to the AS content as an index.
<45> The method or apparatus described in any one of the above items <1> to <29>, wherein the evaluation is carried out for psoriasis by using the ratio of the EOP content to the AS content as an index.
<46> The method or apparatus described in any one of the above items <1> to <45>, wherein it is evaluated that: when the NP/NS ratio of a stratum corneum-derived lipid sample collected from a non-rash part or a healthy part of the arm is 2.7 or higher, the test subject is healthy; when the NP/NS ratio is less than 2.1, the test subject has a possibility of atopic dermatitis; and when the NP/NS ratio is less than 1.6, the test subject has a possibility of psoriasis.
<47> The method or apparatus described in any one of the above items <1> to <45>, wherein it is evaluated that: when the NH/NS ratio of a stratum corneum-derived lipid sample collected from a non-rash part or a healthy part of the arm is 3.2 or higher, the test subject is healthy; when the NH/NS ratio is less than 2.3, the test subject has a possibility of atopic dermatitis; and when the NH/NS ratio is less than 1.5, the test subject has a possibility of psoriasis.
<48> The method or apparatus described in any one of the above items <1> to <45>, wherein it is evaluated that: when the EOH/NS ratio of a stratum corneum-derived lipid sample collected from a non-rash part or a healthy part of the arm is 0.3 or higher, the test subject is healthy; when the EOH/NS ratio is less than 0.3, the test subject has a possibility of atopic dermatitis; and when the EOH/NS ratio is less than 0.2, the test subject has a possibility of psoriasis.
<49> The method or apparatus described in any one of the above items <1> to <45>, wherein it is evaluated that: when the EOP/NS ratio of a stratum corneum-derived lipid sample collected from a non-rash part or a healthy part of the arm is 0.1 or higher, the test subject is healthy; and when the EOP/NS ratio is less than 0.1, the test subject has a possibility of atopic dermatitis or psoriasis.
<50> The method or apparatus described in any one of the above items <1> to <45>, wherein it is evaluated that: when the NP/AS ratio of a stratum corneum-derived lipid sample collected from a non-rash part or a healthy part of the arm is 4.5 or higher, the test subject is healthy; when the NP/AS ratio is less than 2.6, the test subject has a possibility of atopic dermatitis; and when the NP/AS ratio is less than 2.1, the test subject has a possibility of psoriasis.
<51> The method or apparatus described in any one of the above items <1> to <45>, wherein it is evaluated that: when the NH/AS ratio of a stratum corneum-derived lipid sample collected from a non-rash part or a healthy part of the arm is 4.9 or higher, the test subject is healthy; when the NH/AS ratio is less than 2.8, the test subject has a possibility of atopic dermatitis; and when the NH/AS ratio is less than 2.0, the test subject has a possibility of psoriasis.
<52> The method or apparatus described in any one of the above items <1> to <45>, wherein it is evaluated that: when the EOH/AS ratio of a stratum corneum-derived lipid sample collected from a non-rash part or a healthy part of the arm is 0.5 or higher, the test subject is healthy; when the EOH/AS ratio is less than 0.3, the test subject has a possibility of atopic dermatitis; and when the EOH/AS ratio is less than 0.2, the test subject has a possibility of psoriasis.
<53> The method or apparatus described in any one of the above items <1> to <45>, wherein it is evaluated that: when the EOP/AS ratio of a stratum corneum-derived lipid sample collected from a non-rash part or a healthy part of the arm is 2.7 or higher, the test subject is healthy; and when the EOP/AS ratio is less than 0.1, the test subject has a possibility of atopic dermatitis or psoriasis.
<54> The method or apparatus described in any one of the above items <1> to <53>, wherein it is evaluated that prophylaxis of skin disease has been achieved when,

in a case in which the average value of the ceramide content ratio of the healthy group is higher than the average value of the ceramide content ratio of the non-healthy group, at least one ceramide content ratio among the ceramide content ratios described above, and preferably all the ceramide content ratios, are maintained in a state of being higher than the reference value that serves as the evaluation criteria for the health of the skin, and

in a case in which the average value of the ceramide content ratio of the healthy group is lower than the average value of the ceramide content ratio of the non-healthy group, at least one ceramide content ratio among the ceramide content ratios described above, and preferably all the ceramide content ratios, are maintained in a state of being lower than the reference value that serves as the evaluation criteria for the health of the skin.

<55> The method or apparatus described in the above item <54>, wherein when the NP/NS ratio of a stratum corneum-derived lipid sample collected from a non-rash part or a healthy part maintains a value range of 2.1 or higher, it is evaluated that prophylaxis of atopic dermatitis has been achieved.
<56> The method or apparatus described in the above item <54>, wherein when at least one value range of a NH/NS ratio of 2.3 or higher, an EOH/NS ratio of 0.3 or higher, an EOP/NS ratio of 0.1 or higher, a NP/AS ratio of 2.6 or higher, a NH/AS ratio of 2.8 or higher, an EOH/AS ratio of 0.3 or higher, and an EOP/AS ratio of 0.1 or higher, and preferably all the value ranges, of a stratum corneum-derived lipid sample collected from a non-rash part or a healthy part is maintained, it is evaluated that prophylaxis of atopic dermatitis has been achieved.
<57> The method or apparatus described in the above item <54>, wherein when the NP/NS ratio of a stratum corneum-derived lipid sample collected from a non-rash part or a healthy part maintains a value range of 1.6 or higher, it is evaluated that prophylaxis of psoriasis has been achieved.
<58> The method or apparatus described in the above item <54>, wherein when at least one value range of a NH/NS ratio of 1.5 or higher, an EOH/NS ratio of 0.2 or higher, an EOP/NS ratio of 0.1 or higher, a NP/AS ratio of 2.1 or higher, a NH/AS ratio of 2.0 or higher, an EOH/AS ratio of 0.2 or higher, and an EOP/AS ratio of 0.1 or higher, and preferably all the value ranges, of a stratum corneum-derived lipid sample collected from a non-rash part or a healthy part is maintained, it is evaluated that prophylaxis of psoriasis has been achieved.
<59> The method or apparatus described in any one of the above items <1> to <53>, wherein it is evaluated that amelioration of skin disease has been achieved when,

in a case in which the average value of the ceramide content ratio of the healthy group is higher than the average value of the ceramide content ratio of the non-healthy group, at least one ceramide content ratio among the ceramide content ratios described above, and preferably all the content ratios, are in a state of being greater than the reference value that serves as an evaluation criterion for the health of the skin, and

in a case in which the average value of the ceramide content ratio of the healthy group is lower than the average value of the ceramide content ratio of the non-healthy group, at least one ceramide content ratio among the ceramide content ratios described above, and preferably all the ceramide content ratios, are in a state of being smaller than the reference value that serves as an evaluation criterion for the health of the skin.

<60> The method or apparatus described in the above item <59>, wherein when a NP/NS ratio of 2.1 or higher of a stratum corneum-derived lipid sample collected from a non-rash part is attained, it is evaluated that atopic dermatitis has been ameliorated.
<61> The method or apparatus described in the above item <59>, wherein when at least one value range of a NH/NS ratio of 2.3 or higher, an EOH/NS ratio of 0.3 or higher, an EOP/NS ratio of 0.1 or higher, a NP/AS ratio of 2.6 or higher, a NH/AS ratio of 2.8 or higher, an EOH/AS ratio of 0.3 or higher, and an EOP/AS ratio of 0.1 or higher, and preferably all the value ranges, of a stratum corneum-derived lipid sample collected from a non-rash part is attained, it is evaluated that atopic dermatitis has been ameliorated.
<62> The method or apparatus described in the above item <59>, wherein when a NP/NS ratio of 1.6 or higher of a stratum corneum-derived lipid sample collected from a non-rash part is attained, it is evaluated that psoriasis has been ameliorated.
<63> The method or apparatus described in the above item <59>, wherein when at least one value range of a NH/NS ratio of 1.5 or higher, an EOH/NS ratio of 0.2 or higher, an EOP/NS ratio of 0.1 or higher, a NP/AS ratio of 2.1 or higher, a NH/AS ratio of 2.0 or higher, an EOH/AS ratio of 0.2 or higher, and an EOP/AS ratio of 0.1 or higher, and preferably all the value ranges, of a stratum corneum-derived lipid sample collected from a non-rash part is attained, it is evaluated that psoriasis has been ameliorated.
<64> The method or apparatus described in any one of the above items <1> to <27>, wherein a skin quality is evaluated, and preferably any one selected from the group consisting of the skin barrier function, the horny layer water content, the skin color or skin luster, the skin texture, and the desquamation of skin is evaluated, as the health of the skin.
<65> The method or apparatus described in any one of the above items <1> to <27> and <64>, wherein it is considered that: when the NH/NS ratio of a stratum corneum-derived lipid sample collected from a buccal region is 1.5 or higher, the “skin barrier function is normal” or the “skin barrier function is higher than or equal to the average”; and when the NH/NS ratio is less than 1.5, “there is a possibility that the skin barrier function may be abnormal”.
<66> The method or apparatus described in any one of the above items <1> to <27> and <64>, wherein it is considered that: when the EOH/NS ratio of a stratum corneum-derived lipid sample collected from a buccal region is 0.15 or greater, the “horny layer water content is large” or the “horny layer water content is higher than or equal to the average”; and when the EOH/NS ratio is less than 0.15, “there is a possibility that the horny layer water content may be low”.
<67> The method or apparatus described in any one of the above items <1> to <27> and <64>, wherein it is considered that: when the EOP/NS ratio of a stratum corneum-derived lipid sample collected from a buccal region is 0.05 or higher, “desquamation is not at all observed” or “desquamation is slightly observed”; and when the EOP/NS ratio is less than 0.05, “there is a possibility that desquamation may be observed”.
<68> The method or apparatus described in any one of the above items <1> to <27> and <64>, wherein it is considered that: when the NH/NS ratio of a stratum corneum-derived lipid sample collected from a buccal region is 1.6 or higher, the “skin texture is well-ordered” or the “skin texture is fine”; and when the NH/NS ratio is less than 1.6, “there is a possibility that the skin texture may be disordered”.
<69> The method or apparatus described in any one of the above items <1> to <27> and <64>, wherein it is considered that: when the NP/AS ratio of a stratum corneum-derived lipid sample collected from a buccal region is 2.0 or greater, the “skin color is bright” or the “skin has a healthy skin color”; and when the NP/AS ratio is less than 2.0, “there is a possibility that the skin color may be dark” or “there is a possibility that the skin color may not be healthy”.
<70> The method or apparatus described in any one of the above items <1> to <27> and <64>, wherein it is considered that: when the NP/AS ratio of a stratum corneum-derived lipid sample collected from a buccal region is 2.0 or greater, the “skin is less reddish tinged”; and when the NP/AS ratio is less than 2.0, “there is a possibility that the skin may be more reddish tinged”.
<71> A method of screening a prophylactic or ameliorating agent for skin disease or a skin quality improving agent, containing the steps of:

applying a candidate substance for a prophylactic or ameliorating agent for a skin disease (preferably atopic dermatitis or psoriasis) or a skin quality improving agent (preferably an ameliorating agent for at least one selected from the group consisting of skin barrier function, horny layer water content, skin color or skin luster, skin texture, and desquamation of skin; and more preferably an ameliorating agent for skin barrier function, horny layer water content, skin color or skin luster, skin texture, and desquamation of skin) on the skin of a test subject;

checking any change in the health of the skin before and after the application of the candidate substance for a prophylactic or ameliorating agent for a skin disease or a skin quality improving agent by carrying out the method or utilizing the apparatus described in any one of the above items <1> to <70>; and

selecting a substance that provides a prophylactic or ameliorating effect for a skin disease, or a substance that provides a skin quality improving effect, as a prophylactic or ameliorating agent for a skin disease or a skin quality improving agent.

### EXAMPLES

The present invention will be described in more detail based on examples given below, but the invention is not meant to be limited by these.

### Example 1 Correlativity Between Atopic Dermatitis and Ceramide Components

### (1) Test Subject

Eight patients (age of 16 to 36) of atopic dermatitis who regularly visit a dermatologist and seven healthy volunteers (age of 25 to 37) at ages corresponding to the ages of the patients.

### (2) Measurement of Horny Layer Function

For a rash part of the arm and a non-rash part adjacent to the rash part in the patients of atopic dermatitis, and for the same sites in the arm as those of the patient in the healthy person, the object site was washed with a detergent, and then the sites were acclimatized for five minute. Then, measurement of the horny layer water content (Capacitance (AU)) using a corneometer (CORNEOMETER CM825, manufactured by Courage+Khazaka electronic GmbH) and measurement of the transepidermal water loss (TEWL (gm⁻² h⁻¹)) using a tewameter (TEWAMETER TM300, manufactured by Courage+Khazaka Electronic GmbH) were carried out.

### (3) Collection of Stratum Corneum

A tape (PPS tape, manufactured by Nichiban Co., Ltd.) was firmly pressed against the rash part of the arm and the adjacent non-rash part, where measurement of the horny layer functions was measured, in the patients of atopic dermatitis, and against the same sites in the arm of the healthy person as those parts in the patient, and the stratum corneum was peeled off from the same sites successively for ten times (2.5 cm×4 cm×10 sheets). Each of the tape pieces was cut in half, and one of them was submitted to an analysis of ceramide components, while the other one was submitted to quantitative determination of proteins.

### (4) Quantitative Determination of Proteins

A 0.1N solution of sodium hydroxide and a 1% aqueous solution of SDS were added to one piece of the tape that had been cut in half, and the mixture was heated for 2 hours at 60° C. to solubilize proteins. The mixture was cooled to room temperature. Subsequently, 2N hydrochloric acid was added thereto to neutralize the system, and a quantitative value of proteins was obtained by using a BCA Protein Assay (manufactured by Thermo-Fisher Scientific, Inc.) from a calibration curve obtained with BSA.

### (5) Extraction of Lipid Molecules

To the tape with which the stratum corneum had been collected, methanol containing 50 nmol/L N-heptadecanoyl sphingosine as an internal standard substance was added, and the tape was irradiated with ultrasonic waves to extract lipid molecules.

### (6) Crude Fractionation of Ceramide Components and Preparation of Sample Solution

The methanol extract was dried to solid under a nitrogen gas stream, and chloroform/methanol=99.5/0.5 (v/v) was added to this dried methanol extract to dissolve the extract. The solution was applied to a silica gel cartridge for solid phase extraction. Chloroform/methanol=99.5/0.5 (v/v) was sufficiently applied to the cartridge, subsequently chloroform/methanol=95/5 (v/v) was applied to the cartridge, and an eluate thereof was obtained. This eluate was dried to solid under a nitrogen gas stream, subsequently hexane/isopropanol/formic acid=95/5/0.1 (v/v/v) was added thereto to dissolve the eluate, and thus a sample solution was prepared.

### (7) Analysis Conditions for Ceramide Components

As an analysis system in which a liquid chromatograph and a mass analysis apparatus are integrated, AGILENT 1100 series LC/MSD (ESI, single quadrupole, manufactured by Agilent Technologies, Inc.) was used.

As a separation column, INERTSIL SIL 100A-3 (trade name, manufactured by GL Sciences, Inc., 1.5 mmϕ×150 mm (3 μm)) was used. As a guard column, INERTSIL SIL 100A-3 (trade name, manufactured by GL Sciences, Inc., 1.5 mmϕ×10 mm (3 μm)) was used.

As an eluent, two kinds of solutions (eluent A: hexane/isopropanol/formic acid=95/5/0.1 (v/v/v); eluent B: hexane/isopropanol/50 mmol/L aqueous solution of ammonium formate=25/65/10 (v/v/v)) was used. Furthermore, the gradient conditions for eluent A and eluent B are shown in Table 1.

TABLE 1

Time (minute)

| | 0 | 3 | 35 | 40 | 50 | 80 |
|---|---|---|---|---|---|---|
| Eluent A (%) | 100 | 90 | 0 | 0 | 100 | 100 |
| Eluent B (%) | 0 | 10 | 100 | 100 | 0 | 0 |

Flow rate of eluent: 0.1 mL/min
Column temperature: 40° C.
Sample solution injection amount: 10 μL

As an ionization promoter liquid, isopropanol/5 mmol/L ammonium formate-containing methanol solution=50/50 (v/v) was used. The flow rate of the ionization promoter liquid was set to 0.1 mL/min.

Furthermore, the analysis conditions for the mass analysis apparatus were as follows.

Ionization method: ESI

### Polarity: Positive ion

Measurement mass range: 250 to 1,500
Fragmentor voltage: 150 V
Vcap voltage: 3,500 V
Nebulizer pressure: 20 psig
Dry gas temperature: 300° C.
Dry gas flow rate: 8 L/min

The data obtained from the mass analysis apparatus were developed into a multi-stage mass chromatogram having three axes of retention time, m/z, and ionic strength. Subsequently, various peaks included in the multi-stage mass chromatogram were identified by utilizing a database storing the respective information of retention time and m/z for known ceramide molecular species. Then, the peak area of each of the ceramide molecules was determined, the peak area ratio with respect to the internal standard substance was calculated, and the peak area ratio was divided by the amount of proteins. Thereby, the relative amount of each ceramide molecule per unit amount of proteins was calculated. This was multiplied by the detection sensitivity correction coefficient for each ceramide molecular species that had been determined in advance, and thereby the proportion (%) of the absolute amount of each ceramide molecular species with respect to the total amount (absolute amount) of all ceramides per unit amount of proteins was calculated.

Furthermore, the content ratios from the absolute amounts of various ceramide molecular species per unit amount of proteins for the ceramide component A and the ceramide component B (NP/NS ratio, NH/NS ratio, EOH/NS ratio, EOP/NS ratio, NP/AS ratio, NH/AS ratio, EOH/AS ratio, and EOP/AS ratio) were calculated.

### (8) Calculation of Correlation Coefficient Between Quantitative Value of Ceramide and Horny Layer Functions of Rash Part and Non-Rash Part of Atopic Dermatitis and Healthy Part of Healthy Person

The Pearson correlation coefficient between the absolute amounts of various ceramide molecular species per unit amount of proteins, the proportions of various ceramide molecular species in the total amount of all ceramides or the ratio of the content of ceramide component A and the content of ceramide component B calculated in section (7), and the horny layer water content (Capacitance) or the transepidermal water loss (TEWL) measured in section (2) were calculated. A p value of less than 0.05 was judged to be meaningful.

### (9) Comparison Between Quantitative Values of Rash Part and Non-Rash Part of Atopic Dermatitis and Healthy Part of Healthy Person

A comparison of the absolute amounts of various ceramide molecular species per unit amount of proteins, the proportions of various ceramide molecular species in the total amount of all ceramides, and the ratio of the content of ceramide component A and the content of ceramide component B calculated in section (7) was made between three groups, namely, rash parts and non-rash parts of atopic dermatitis, and healthy parts of healthy persons. Bonferroni's multiple comparative test was carried out, and a p value of less than 0.05 was judged to be meaningful.

The results are shown in Table 2. Meanwhile, in the following Table 2 and Table 3 described below, the expression “NP/NS” represents the ratio of the NP content to the NS content. The expression “NH/NS” represents the ratio of the NH content to the NS content. The expression “EOH/NS” represents the ratio of the EOH content to the NS content. The expression “EOP/NS” represents the ratio of the EOP content to the NS content. The expression “NP/AS” represents the ratio of the NP content to the AS content. The expression “NH/AS” represents the ratio of the NH content to the AS content. The expression “EOH/AS” represents the ratio of the EOH content to the AS content. The expression “EOP/AS” represents the ratio of the EOP content to the AS content.

TABLE 2

| | | | | | | | | | | Rash part vs Non-rash | | |
|---|---|---|---|---|---|---|---|---|---|---|---|---|
| | | Atopic dermatitis | | Atopic dermatitis | | | | Correlation | | part vs Healty | | |
| | | Rash part | | Non-rash part | | Healthy | | coefficient | | Rash vs | Rash vs | Non-rash |
| | | Mean | SD | Mean | SD | Mean | SD | TEWL | Capacitance | Non-rash | Healty | vs Healty |
| ng/μg | NDS | 1.3 | 0.4 | 1.7 | 0.3 | 1.7 | 0.4 | −0.64 | 0.61 | * | NS | NS |
| protein | NS | 2.4 | 0.9 | 2.2 | 0.4 | 2.0 | 0.4 | 0.28 | −0.23 | NS | NS | NS |
| | NH | 3.7 | 1.9 | 5.8 | 1.4 | 7.6 | 1.8 | −0.73 | 0.61 | * | ** | NS |
| | NP | 3.2 | 1.7 | 5.4 | 1.6 | 6.7 | 1.5 | −0.74 | 0.63 | * | ** | NS |
| | ADS | 0.3 | 0.1 | 0.4 | 0.1 | 0.3 | 0.1 | −0.38 | 0.42 | NS | NS | NS |
| | AS | 2.1 | 0.7 | 1.7 | 0.3 | 1.2 | 0.3 | 0.45 | −0.36 | NS | ** | NS |
| | AH | 3.5 | 1.5 | 5.0 | 1.2 | 5.0 | 1.4 | −0.70 | 0.67 | NS | NS | NS |
| | AP | 4.8 | 1.6 | 5.6 | 1.5 | 4.7 | 1.9 | −0.44 | 0.51 | NS | NS | NS |
| | EOS | 0.9 | 0.4 | 1.3 | 0.5 | 1.5 | 0.5 | −0.59 | 0.57 | NS | * | NS |
| | EOH | 0.3 | 0.2 | 0.5 | 0.2 | 0.7 | 0.2 | −0.68 | 0.67 | NS | ** | NS |
| | EOP | 0.1 | 0.1 | 0.2 | 0.1 | 0.3 | 0.1 | −0.69 | 0.66 | NS | ** | NS |
| | Total | 22.8 | 7.9 | 30.0 | 6.6 | 31.6 | 7.1 | −0.67 | 0.63 | NS | * | NS |
| % | NDS | 5.9 | 0.8 | 5.8 | 0.7 | 5.4 | 0.4 | 0.37 | −0.33 | NS | NS | NS |
| | NS | 11.1 | 4.6 | 7.4 | 1.1 | 6.5 | 1.1 | 0.87 | −0.69 | * | ** | NS |
| | NH | 15.7 | 2.5 | 19.4 | 2.0 | 24.0 | 3.0 | −0.67 | 0.47 | * | ** | ** |
| | NP | 13.7 | 3.7 | 17.7 | 2.4 | 21.3 | 2.5 | −0.70 | 0.51 | * | ** | * |
| | ADS | 1.4 | 0.3 | 1.3 | 0.3 | 1.1 | 0.1 | 0.44 | −0.34 | NS | * | NS |
| | AS | 9.7 | 3.7 | 5.9 | 0.7 | 3.9 | 0.6 | 0.89 | −0.72 | ** | ** | NS |
| | AH | 15.1 | 1.8 | 16.7 | 1.1 | 15.7 | 1.9 | −0.54 | 0.50 | NS | NS | NS |
| | AP | 21.4 | 2.9 | 18.7 | 2.6 | 14.4 | 4.1 | 0.29 | −0.17 | NS | ** | * |
| | EOS | 3.9 | 1.4 | 4.5 | 1.5 | 4.8 | 0.9 | −0.29 | 0.27 | NS | NS | NS |
| | EOH | 1.4 | 0.6 | 1.8 | 0.5 | 2.2 | 0.3 | −0.57 | 0.53 | NS | * | NS |
| | EOP | 0.6 | 0.2 | 0.7 | 0.2 | 0.9 | 0.1 | −0.68 | 0.61 | * | ** | * |
| ratio | NP/NS | 1.4 | 0.7 | 2.4 | 0.5 | 3.4 | 0.7 | −0.74 | 0.65 | ** | ** | * |
| | NH/NS | 1.6 | 0.7 | 2.7 | 0.3 | 3.8 | 0.6 | −0.77 | 0.65 | ** | ** | ** |
| | EOH/NS | 0.2 | 0.1 | 0.2 | 0.1 | 0.3 | 0.1 | −0.68 | 0.66 | NS | ** | NS |
| | EOP/NS | 0.1 | 0.0 | 0.1 | 0.0 | 0.1 | 0.0 | −0.68 | 0.65 | * | ** | * |
| | NP/AS | 1.7 | 0.9 | 3.1 | 0.7 | 5.7 | 1.2 | −0.64 | 0.50 | * | ** | ** |
| | NH/AS | 1.9 | 0.9 | 3.3 | 0.6 | 6.4 | 1.5 | −0.62 | 0.48 | * | ** | ** |
| | EOH/AS | 0.2 | 0.1 | 0.3 | 0.1 | 0.6 | 0.1 | −0.64 | 0.54 | * | ** | ** |
| | EOP/AS | 0.1 | 0.0 | 0.1 | 0.0 | 0.2 | 0.0 | −0.66 | 0.56 | ** | ** | ** |

*: p < 0.05,
**: p < 0.01 by Bonferroni

The following abbreviations in Table 2 described above and Table 3 that will be described below respectively refer to the following ceramides.

NDS: non-hydroxyacyl-dihydrosphingosine ceramide (referring to a ceramide having a structure in which a dihydroxysphingosine and a non-hydroxy fatty acid are bonded by an amide bond.)
ADS: α-hydroxyacyl-dihydrosphingosine ceramide (referring to a ceramide having a structure in which a dihydroxysphingosine and an α-hydroxy fatty acid are bonded by an amide bond.)
AH: α-hydroxyacyl-6-hydroxysphingosine ceramide (referring to a ceramide having a structure in which a 6-hydroxysphingosine and an α-hydroxy fatty acid are bonded by an amide bond.)
AP: α-hydroxyacyl-phytosphingosine ceramide (referring to a ceramide having a structure in which a phytosphingosine and an α-hydroxy fatty acid are bonded by an amide bond.)
EOS: esterified w-hydroxyacyl-sphingosine ceramide (referring to a ceramide having a structure in which a sphingosine and an esterified ω-hydroxy fatty acid are bonded by an amide bond.)

As shown in Table 2, the ceramide class composition of a rash part of an atopic dermatitis patient is largely different from the ceramide class composition of a healthy person. However, in the ceramide class composition of a non-rash part of an atopic dermatitis patient and the ceramide class composition of a healthy person, significant differences were recognized only in the proportion (%) of the absolute amount of the NH, the proportion (%) of the absolute amount of the NP, the proportion (%) of the absolute amount of the AP, and the proportion (%) of the absolute amount of the EOP.

In contrast, among the indices of the present invention, in regard to NP/NS, NH/NS, EOP/NS, NP/AS, NH/AS, EOH/AS and EOP/AS, significant differences were recognized between a non-rash part of an atopic dermatitis patient and a healthy person. Further, in addition to these content ratios, high correlativity of EOH/NS with respect to the horny layer water content or the transepidermal water loss was also recognized.

### Example 2 Correlativity Between Psoriasis and Ceramide Components

### (1) Test Subject

Eight patients (age of 36 to 74) of psoriasis who regularly visit a dermatologist and nine healthy volunteers (age of 39 to 76) at ages corresponding to the ages of the patients.

### (2) Measurement of Horny Layer Function

For a rash part of the arm and a non-rash part adjacent to the rash part in the patients of psoriasis, and for the same sites in the arm as those of the patient in the healthy person, measurement of the horny layer water content and measurement of the transepidermal water loss were carried out in the same manner as in Example 1.

### (3) Collection of Stratum Corneum

A tape (PPS tape, manufactured by Nichiban Co., Ltd.) was firmly pressed against the rash part of the arm and the adjacent non-rash part, where measurement of the horny layer functions was measured, in the patient of psoriasis, and against the same sites in the arm of the healthy person as those parts in the patient, and the stratum corneum was peeled off from the same sites successively for ten times (2.5 cm×4 cm×10 sheets). Each of the tape pieces was cut in half, and one of them was submitted to an analysis of ceramide components, while the other one was submitted to quantitative determination of proteins.

### (4) Quantitative Determination of Proteins

Quantitative determination of proteins was carried out in the same manner as in Example 1 by using a tape that was cut in half.

### (5) Analysis of Ceramide Components

The absolute amounts of various ceramide molecular species per unit amount of proteins, the proportions (%) of the absolute amounts of various ceramide molecular species with respect to the total amount (absolute amount) of all ceramides per unit amount of proteins, and the NP/NS ratio, the NH/NS ratio, the EOH/NS ratio, the EOP/NS ratio, the NP/AS ratio, the NH/AS ratio, the EOH/AS ratio and the EOP/AS ratio were calculated in the same manner as in Example 1, by using tapes with which the stratum corneum had been collected. Thus, the correlation coefficient to the horny layer functions of a rash part and a non-rash part of psoriasis and a healthy part of a healthy person, and the “comparison of quantitative values of ceramides between a rash part and a non-rash part of psoriasis and a healthy part of a healthy person” were calculated.

The results are presented in Table 3.

TABLE 3

| | | | | | | | | | | Rash part vs Non-rash | | |
|---|---|---|---|---|---|---|---|---|---|---|---|---|
| | | Psoriasis | | Psoriasis | | | | | | part vs Healty | | |
| | | Rash part | | Non-rash part | | Healthy | | Correlation coefficient | | Rash vs | Rash vs | Non-rash |
| | | Mean | SD | Mean | SD | Mean | SD | TEWL | Capacitance | Non-rash | Healty | vs Healty |
| ng/μg | NDS | 1.6 | 0.7 | 1.9 | 0.7 | 2.3 | 0.6 | −0.32 | 0.51 | NS | NS | NS |
| protein | NS | 3.9 | 1.5 | 2.5 | 0.7 | 2.4 | 0.7 | 0.28 | −0.33 | ** | ** | NS |
| | NH | 3.7 | 1.5 | 5.4 | 1.1 | 7.1 | 1.7 | −0.39 | 0.77 | NS | ** | NS |
| | NP | 2.9 | 1.8 | 6.1 | 1.5 | 7.7 | 2.4 | −0.51 | 0.76 | ** | ** | NS |
| | ADS | 0.5 | 0.2 | 0.5 | 0.1 | 0.6 | 0.2 | −0.21 | 0.36 | NS | NS | NS |
| | AS | 3.1 | 1.2 | 1.8 | 0.5 | 1.5 | 0.7 | 0.35 | −0.40 | ** | ** | NS |
| | AH | 3.8 | 1.7 | 4.8 | 1.0 | 5.5 | 1.7 | −0.28 | 0.62 | NS | * | NS |
| | AP | 2.9 | 1.3 | 4.4 | 0.9 | 4.8 | 2.1 | −0.36 | 0.64 | NS | * | NS |
| | EOS | 0.7 | 0.4 | 1.2 | 0.5 | 1.4 | 0.5 | −0.51 | 0.71 | * | * | NS |
| | EOH | 0.2 | 0.2 | 0.5 | 0.2 | 0.6 | 0.2 | −0.60 | 0.73 | * | ** | NS |
| | EOP | 0.1 | 0.1 | 0.2 | 0.1 | 0.3 | 0.1 | −0.57 | 0.72 | ** | ** | NS |
| % | Total | 23.4 | 8.8 | 29.1 | 5.9 | 34.1 | 9.4 | −0.35 | 0.66 | NS | * | NS |
| | NDS | 6.9 | 1.9 | 6.4 | 1.5 | 6.6 | 0.8 | 0.00 | −0.17 | NS | NS | NS |
| | NS | 17.5 | 5.1 | 8.5 | 1.5 | 7.1 | 1.9 | 0.59 | −0.77 | ** | ** | NS |
| | NH | 15.5 | 1.7 | 18.4 | 1.8 | 21.0 | 2.0 | −0.41 | 0.66 | ** | ** | ** |
| | NP | 12.1 | 5.3 | 20.8 | 2.6 | 22.5 | 3.5 | −0.57 | 0.69 | ** | ** | NS |
| | ADS | 2.0 | 0.5 | 1.6 | 0.3 | 1.6 | 0.3 | 0.15 | −0.43 | NS | * | NS |
| | AS | 13.8 | 4.3 | 6.2 | 1.4 | 4.5 | 1.1 | 0.60 | −0.78 | ** | ** | NS |
| | AH | 15.9 | 1.6 | 16.5 | 1.9 | 16.2 | 1.7 | 0.01 | 0.12 | NS | NS | NS |
| | AP | 12.3 | 2.8 | 15.1 | 2.3 | 13.8 | 3.3 | −0.22 | 0.33 | NS | NS | NS |
| | EOS | 3.0 | 0.9 | 4.1 | 1.3 | 4.1 | 1.5 | −0.44 | 0.52 | NS | NS | NS |
| | EOH | 0.8 | 0.4 | 1.5 | 0.5 | 1.8 | 0.6 | −0.58 | 0.67 | ** | ** | NS |
| | EOP | 0.3 | 0.3 | 0.7 | 0.3 | 0.8 | 0.2 | −0.61 | 0.67 | ** | ** | NS |
| ratio | NP/NS | 0.9 | 0.7 | 2.5 | 0.7 | 3.4 | 1.2 | −0.57 | 0.64 | ** | ** | * |
| | NH/NS | 1.0 | 0.5 | 2.2 | 0.5 | 3.1 | 0.6 | −0.55 | 0.70 | ** | ** | ** |
| | EOH/NS | 0.1 | 0.1 | 0.2 | 0.1 | 0.3 | 0.1 | −0.62 | 0.68 | ** | ** | NS |
| | EOP/NS | 0.0 | 0.0 | 0.1 | 0.0 | 0.1 | 0.0 | −0.61 | 0.65 | ** | ** | NS |
| | NP/AS | 1.1 | 1.0 | 3.5 | 1.1 | 5.4 | 1.8 | −0.56 | 0.62 | ** | ** | * |
| | NH/AS | 1.3 | 0.7 | 3.1 | 0.9 | 4.9 | 1.0 | −0.53 | 0.67 | ** | ** | ** |
| | EOH/AS | 0.1 | 0.1 | 0.3 | 0.1 | 0.4 | 0.1 | −0.56 | 0.68 | ** | ** | * |
| | EOP/AS | 0.0 | 0.0 | 0.1 | 0.1 | 0.2 | 0.1 | −0.59 | 0.65 | ** | ** | * |

*: p < 0.05.
**: p < 0.01 by Bonferroni

As shown in Table 3, the ceramide class composition of a rash part of a psoriasis patient is largely different from the ceramide class composition of a healthy person. However, no significant difference was recognized between the ceramide class composition of a non-rash part of a psoriasis and the ceramide class composition of a healthy person, except for the proportion (%) of the absolute amount of the NH.

In contrast, among the indices of the present invention, in regard to NP/NS, NH/NS, NP/AS, NH/AS, EOH/AS and EOP/AS, significant differences were recognized between a non-rash part of a psoriasis patient and a healthy person. Further, high correlativity of these content ratios with respect to the horny layer water content or the transepidermal water loss was also recognized.

### Example 3 Correlativity Between Skin Quality and Ceramide Components

### (1) Test Subject

Two-hundred and ten healthy women in total (average age 45.9) ranging from the early 20's to the early 70's and living in areas around Tokyo.

### (2) Collection of Stratum Corneum

From the buccal region of each test subject, the stratum corneum was collected successively for four times from the same site by a tape stripping method (2.5 cm×4 cm×four sheets). As the tape, an acrylic adhesive tape (manufactured by Teraoka Seisakusho Co., Ltd.) was used. Each sheet of tape was cut in half, and one of them was submitted to an analysis of ceramide components, while the other one was submitted to quantitative determination of proteins. Regarding quantitative determination of proteins, a 0.1N aqueous solution of NaOH and a 1% aqueous solution of SDS were added to one piece of the tape that had been cut in half, and the mixture was heated for 2 hours at 60° C. to solubilize proteins. The mixture was cooled to room temperature. Subsequently, 2N hydrochloric acid was added thereto to neutralize the system, and a quantitative value of proteins was obtained by using a BCA Protein Assay (manufactured by Thermo-Fisher Scientific, Inc.) from a calibration curve obtained with BSA.

### (3) Preparation of Lipid Sample

The tape with which the stratum corneum was collected was immersed in 1.9 mL of methanol in a 5-mL screwed tube (Maruemu Corp.; No. 2) and was ultrasonically treated for 10 minutes at room temperature, and thus lipids were extracted. Subsequently, 100 μL of a methanol solution containing an internal standard (N-heptadecanoyl-D-erythro-sphingosine) was added to this screwed tube, and a lipid solution was prepared.

### (4) Quantitative Determination of Content of Ceramide Component a and Content of Ceramide Component B, and Calculation of Ratio of Content of Ceramide Component a to Content of Ceramide Component B

Quantitative determination of the content of ceramide component A and the content of ceramide component B included in the lipid sample was carried out using a liquid chromatograph-mass analysis apparatus (manufactured by Agilent Technologies, Inc., LC/Multi ion source-MS). As a separation column, L-column ODS 2.1 mm i.d.×150 mm (5 μm) was used.

As an eluent, two kinds of solutions (eluent A: a 50% methanol solution containing 10 mmol/L ammonium acetate; eluent B: a 2-propanol solution containing 10 mmol/L ammonium acetate) were used. Furthermore, the gradient conditions for the eluents A and B are presented in Table 4.

TABLE 4

| | Time (minute) | | | | | | |
|---|---|---|---|---|---|---|---|
| | 0 | 1 | 2 | 30 | 35 | 35.1 | 45 |
| Eluent A (%) | 80 | 80 | 40 | 0 | 0 | 80 | 80 |
| Eluent B (%) | 20 | 20 | 60 | 100 | 100 | 20 | 20 |

The conditions for analysis by the mass analysis apparatus were as follows.

Ion source: Multi-mode ion source
Ionization method: ESI method
Detection mode: SIM detection of acetate ion-added molecules of ceramide
([M⁺CH₃COO]⁻) in a negative ion mode
Dry gas flow rate: 4 L/min
Nebulizer pressure=60 psig
Dry gas temperature: 350° C.
Vaporizer temperature: 200° C.
Capillary voltage: 4,000 V
Charging voltage: 2,000 V

The data obtained from the mass analysis apparatus were developed into a multi-stage mass chromatogram having three axes of retention time, m/z, and ionic strength. Subsequently, peaks originating from ceramide component A and ceramide component B among the various peaks included in the multi-stage mass chromatogram were identified by utilizing a database storing the respective information of retention time and m/z for known ceramide molecular species.

Then, the peak areas of the peaks originating from ceramide component A and ceramide component B were determined, the peak area ratios with respect to the internal standard substance were calculated, and the relative amounts of the peak originating from ceramide component A and ceramide component B were calculated. The calculated values were multiplied by the detection sensitivity correction coefficient of each of the ceramide components A and ceramide components B that had been determined in advance, and the resultant was divided by the amount of proteins. Thus, the absolute amounts of ceramide component A and ceramide component B per unit amount of proteins, and the ratio of the content of ceramide component A to the content of ceramide component B were calculated.

### (5) Measurement of Skin Quality

The buccal region of a test subject, for whom the skin quality was to be evaluated, was cleaned, and the skin of the region was acclimatized for 30 minutes in an environment at 24° C. and a humidity of 40% and was further acclimatized for 5 minutes in an environment at 20° C. and a humidity of 40%. Subsequently, measurement of various skin qualities as described below was carried out.

### (i) Skin Color and Skin Luster

A sensor was brought into contact with a cheek using a spectral colorimeter (CM2002, manufactured by Konica Minolta, Inc.) under the conditions of a C light source and a 2-degree viewing field, and the skin color and skin luster (L* value (AU) and a* value (AU)) were measured. Measurement was made five times at the same site. The maximum value and the minimum value were rejected, and the average value of three results was calculated.

### (ii) Skin Barrier Function

A tewameter (TEWAMETER TM300, manufactured by Courage+Khazaka Electronic GmbH) was brought into contact with a cheek, and the transepidermal water loss (TEWL (gm⁻²h⁻¹)) was measured. Measurement was made three times at the same site, and the average value was calculated. Meanwhile, the measurement of the transepidermal water loss was set so as to be terminated when the standard deviation of the measured value was within the range of 0.1.

(iii) Horny Layer Water Content

A sensor was firmly pressed against a cheek using a corneometer (CORNEOMETER CM825, manufactured by Courage+Khazaka Electronic GmbH), and the horny layer water content (Capacitance (AU)) was measured. Measurement was made five times at the same site. Then, the maximum value and the minimum value were rejected, and the average value of three results was calculated.

### (iv) Skin Texture

A photograph of a cheek was taken using a 50×PL lens of a skin scope (i-SCOPE USB 2.0, manufactured by Moritex Corp.). Then, the skin texture of the cheek was scored from the photograph thus taken, based on the skin texture score scale shown in FIG. 2 (evaluation in seven grades from 1.0 to 4.0).

### (iv) Desquamation of Skin

The degree of desquamation was scored from the photograph taken in section (iv), based on the following evaluation criteria (four grades from 0 to 3).

### <Evaluation Criteria for Desquamation>

0: No desquamation was observed.
1: Slight desquamation was observed.
2: Desquamation was observed.
3: Significant desquamation was observed.

### (6) Calculation of Correlation Coefficient Between Ratio of Content of Ceramide Component a to Content of Ceramide Component B, and Skin Quality

Between the absolute amounts of the content of ceramide component A and the content of ceramide component B calculated in section (4), or the ratio of the content of ceramide component A to the content of ceramide component B, and various property values of the skin quality measured in section (5), Spearman's correlation coefficient was calculated. Meanwhile, a p value of less than 0.05 was judged to be meaningful.

The results are presented in Table 5.

TABLE 5

| | | | | Horny layer | | |
|---|---|---|---|---|---|---|
| | Skin color | | | water content | Skin texture | Desquamation |
| | L* | a* | TEWL | (Capacitance) | score | score |
| NH/NS | 0.130 | −0.157 | −0.445 | 0.177 | 0.273 | −0.135 |
| | | (p < 0.05) | (p < 0.001) | (p < 0.05) | (p < 0.001) | (p < 0.05) |
| EOH/NS | 0.105 | −0.050 | −0.341 | 0.335 | 0.167 | −0.090 |
| | | | (p < 0.001) | (p < 0.001) | (p < 0.05) | |
| EOP/NS | 0.135 | −0.150 | −0.190 | 0.215 | 0.234 | −0.166 |
| | | (p < 0.05) | (p < 0.01) | (p < 0.01) | (p < 0.001) | (p < 0.05) |
| NP/AS | 0.157 | −0.204 | −0.338 | 0.141 | 0.260 | −0.163 |
| | (p < 0.05) | (p < 0.01) | (p < 0.001) | (p < 0.05) | (p < 0.001) | (p < 0.05) |
| NH/AS | 0.059 | −0.132 | −0.379 | 0.131 | 0.191 | −0.104 |
| | | | (p < 0.001) | | (p < 0.01) | |
| EOH/AS | 0.070 | −0.075 | −0.378 | 0.298 | 0.147 | −0.093 |
| | | | (p < 0.001) | (p < 0.001) | (p < 0.05) | |
| EOP/AS | 0.087 | −0.145 | −0.236 | 0.202 | 0.195 | −0.164 |
| | | (p < 0.05) | (p < 0.001) | (p < 0.01) | (p < 0.01) | (p < 0.05) |

As shown in Table 5, the ratio of the content of ceramide component A to the content of ceramide component B (content of ceramide component A/content of ceramide component B) exhibited positive correlativity with the L* value, the horny layer water content, and the skin texture score. Meanwhile, the ratio of the content of ceramide component A to the content of ceramide component B (content of ceramide component A/content of ceramide component B) exhibited negative correlativity with the a* value, the TEWL value, and the desquamation score.

Particularly, the NH/NS ratio exhibited meaningful correlativity with the a* value, the TEWL value, the horny layer water content, the skin texture score, and the desquamation score.

The EOH/NS ratio exhibited meaningful correlativity with the TEWL value, the horny layer water content, and the skin texture score.

The EOP/NS ratio exhibited meaningful correlativity with the a* value, the TEWL value, the horny layer water content, the skin texture score, and the desquamation score.

The NP/AS ratio exhibited meaningful correlativity with the L* value, the a* value, the TEWL value, the horny layer water content, the skin texture score, and the desquamation score.

The NH/AS ratio exhibited meaningful correlativity with the TEWL value, and the skin texture score.

The EOH/AS ratio exhibited meaningful correlativity with the TEWL value, the horny layer water content, and the skin texture score.

The EOP/AS ratio exhibited meaningful correlativity with the a* value, the TEWL value, the horny layer water content, the skin texture score, and the desquamation score.

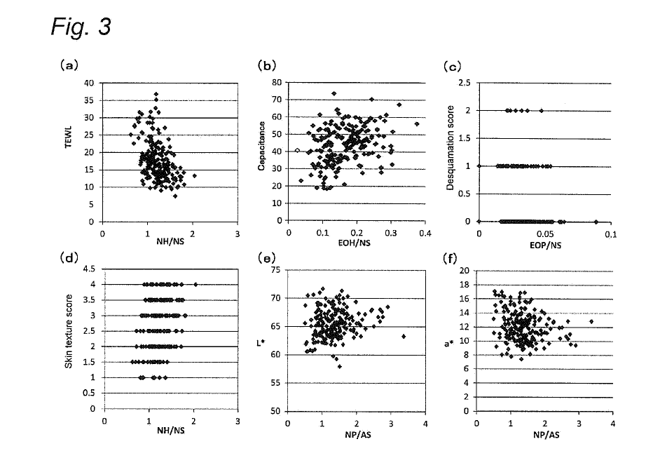

Furthermore, as a specific example, a graph obtained by plotting the TEWL value and the NH/NS ratio of various test subjects is shown in FIG. 3(a). Furthermore, a graph obtained by plotting the Capacitance and the EOH/NS ratio is shown in FIG. 3(b). Furthermore, a graph obtained by plotting the desquamation score and the EOP/NS ratio is shown in FIG. 3(c). A graph obtained by plotting the skin texture score and the NH/NS ratio is shown in FIG. 3(d). A graph obtained by plotting the L* value and the NP/AS ratio is shown in FIG. 3(e). Furthermore, a graph obtained by plotting the a* value and the NP/AS ratio is shown in FIG. 3(f).

As shown in FIG. 3(a), in test subjects having a TEWL value of more than 20, a case in which the NH/NS ratio was 1.5 or higher was almost not observed. Therefore, when the NH/NS ratio is 1.5 or higher, it can be evaluated that the “skin barrier function is normal” or the “skin barrier function is higher than or equal to the average”.

As shown in FIG. 3(b), in test subjects having a horny layer water content (Capacitance) of more than 60, the EOH/NS ratio was approximately 0.15 or higher. Therefore, when the EOH/NS ratio is 0.15 or higher, it can be evaluated that the “horny layer water content is high” or the “horny layer content is higher than or equal to the average”.

As shown in FIG. 3(c), in test subjects having a desquamation score of 2, the EOP/NS ratio was less than 0.05 in all cases. Therefore, when the EOP/NS ratio is 0.05 or higher, it can be evaluated that “desquamation cannot be seen” or “slight desquamation is seen”.

As shown in FIG. 3(d), in test subjects having a NH/NS ratio of 1.6 or higher, the skin texture score was 2.5 or higher in most cases, and the skin texture was well-ordered. Therefore, when the NH/NS ratio is 1.6 or higher, it can be evaluated that the “skin texture is well-ordered” or the “skin texture is fine”.

As shown in FIG. 3(e), in test subjects having a NP/AS ratio of 2.0 or higher, the L* value was approximately 65 or higher. Therefore, when the NP/AS ratio is 2.0 or higher, it can be evaluated that the “skin color is bright” or the “test subject has a healthy skin color”.

As shown in FIG. 3(f), in test subjects having an a* value of 14 or higher, the NP/AS ratio was approximately less than 2.0. Therefore, when the NP/AS ratio is 2.0 or higher, it can be evaluated that the “skin is less reddish tinged”.

From the results of Table 5 and FIGS. 3(a) to 3(f), it was found that the ratio of the content of ceramide component A to the content of ceramide component B has high correlativity with the skin quality. These results show that the ratio of the content of ceramide component A to the content of ceramide component B is an index that detects the correlativity with more skin property values including the skin color precisely and accurately. Furthermore, by evaluating the skin quality using the ratio of the content of ceramide component A to the content of ceramide component B, there is an advantage that calculation of the absolute amounts of various ceramide molecular species based on correction with the amount of proteins or the like is not needed. That is, by employing the ratio of the content of ceramide component A to the content of ceramide component B as an index, the skin quality can be evaluated conveniently and accurately from various viewpoints.

As described above, the health of the skin can be evaluated conveniently and accurately by employing the ratio of the content of ceramide component A to the content of ceramide component B, which are included in the stratum corneum.

Having described our invention as related to the present embodiments, it is our intention that the invention not be limited by any of the details of the description, unless otherwise specified, but rather be construed broadly within its spirit and scope as set out in the accompanying claims.

This application claims priority on Patent Application No. 2016-067643 filed in Japan on Mar. 30, 2016, which is entirely herein incorporated by reference.

### DESCRIPTION OF SYMBOLS

1 Analysis system performing quantitative determination of ceramide component A and ceramide component B, calculation of the ratio of the content of ceramide component A to the content of ceramide component B, and evaluation of the health of the skin of a test subject from the content ratio thus calculated
10 Liquid chromatograph
   11a, 11b Gradient pump
   12 Auto-injector
   13 Guard column
   14 Separation column
   a, b Eluent
   d Lipid sample solution
20 Ionization promoter liquid delivery apparatus
   21 Pump
   22 Connector
   c Ionization promoter liquid
30 Mass analysis apparatus
   31 Ionization apparatus
   32 Mass separation detection apparatus
40 Arithmetic unit

1. A method of evaluating the health of skin, comprising the steps of:
quantitatively determining respective contents of a non-hydroxyacyl-phytosphingosine ceramide component and a non-hydroxyacyl-sphingosine ceramide component included in a lipid sample prepared from a collected sample of a stratum corneum of a test subject;
calculating the content ratio of the quantitatively determined content of the non-hydroxyacyl-phytosphingosine ceramide component to the quantitatively determined content of the non-hydroxyacyl-sphingosine ceramide component; and
evaluating the skin condition for skin disease of the test subject from the calculated content ratio.

2. A method of evaluating the health of skin, comprising the steps of:
quantitatively determining respective contents of one kind of ceramide component A and one kind of ceramide component B included in a lipid sample prepared from a collected sample of a stratum corneum of a test subject;
calculating the content ratio of the quantitatively determined content of the ceramide component A to the quantitatively determined content of the ceramide component B; and
evaluating the health of the skin of the test subject from the calculated content ratio; wherein the ceramide component A is selected from the group consisting of a non-hydroxyacyl-phytosphingosine ceramide component, a non-hydroxyacyl-6-hydroxysphingosine ceramide component, an esterified ω-hydroxyacyl-6-hydroxysphingosine ceramide component, and an esterified ω-hydroxyacyl-phytosphingosine ceramide component; and the ceramide component B is selected from the group consisting of a non-hydroxyacyl-sphingosine ceramide component and an α-hydroxyacyl-sphingosine ceramide component; provided that the case of selecting the non-hydroxyacyl-phytosphingosine ceramide component as the ceramide component A and the non-hydroxyacyl-sphingosine ceramide component as the ceramide component B is excluded.

3.-4. (canceled)

5. The method according to claim 1, wherein the skin disease is atopic dermatitis or psoriasis.

6. The method according to claim 1, wherein the lipid sample is a lipid sample prepared from a collected sample of the stratum corneum from a non-rash part of a test subject or from a healthy part of a test subject who has not developed skin disease.

7. The method according to claim 1, wherein the health of the skin is evaluated, based on the association function between the information of the ratio of the content of the non-hydroxyacyl-phytosphingosine ceramide component, to the content of the non-hydroxyacyl-sphingosine ceramide component, both ceramide components being included in the lipid sample prepared from a collected sample of a stratum corneum, and the health condition of the skin, from the ratio of the quantitatively determined content of the non-hydroxyacyl-phytosphingosine ceramide component to the quantitatively determined content of the non-hydroxyacyl-sphingosine ceramide component.

8. The method according to claim 1, wherein the presence or absence of the onset of atopic dermatitis or psoriasis, the possibility of the onset of atopic dermatitis or psoriasis, the prophylactic state of atopic dermatitis or psoriasis, the progress of atopic dermatitis or psoriasis, the presence or absence of a tendency (predisposition) toward atopic dermatitis or psoriasis, the status of healing of atopic dermatitis or psoriasis, or therapeutic effects for atopic dermatitis or psoriasis, is evaluated as the skin health.

9. (canceled)

10. The method according to claim 2, wherein the presence or absence of the onset of atopic dermatitis or psoriasis, the possibility of the onset of atopic dermatitis or psoriasis, the prophylactic state of atopic dermatitis or psoriasis, the progress of atopic dermatitis or psoriasis, the presence or absence of a tendency (predisposition) toward atopic dermatitis or psoriasis, the status of healing of atopic dermatitis or psoriasis, or therapeutic effects for atopic dermatitis or psoriasis, or at least one selected from the group consisting of the skin barrier function, the horny layer water content, the skin color or skin luster, the skin texture, and the desquamation of skin is evaluated as the health of skin quality.

11. (canceled)

12. The method according to claim 1, wherein in a case in which the average value of the ceramide content ratio of the healthy group is higher than the average value of the ceramide content ratio of the non-healthy group,
when the ceramide content ratio thus calculated is higher than the lower limit of the value range of the ceramide content ratio characterizing the healthy group or the upper limit of the value range of the ceramide content ratio characterizing the non-healthy group, the test subject is evaluated as “being healthy”, and
when the ceramide content ratio thus calculated is lower than or equal to the lower limit of the value range of the ceramide content ratio characterizing the healthy group or the upper limit of the value range of the ceramide content ratio characterizing the non-healthy group, the test subject is evaluated as “having a possibility of being non-healthy” or “having a high possibility of being non-healthy”; and
wherein in a case in which the average value of the ceramide content ratio of the healthy group is lower than the average value of the ceramide content ratio of the non-healthy group,
when the ceramide content ratio thus calculated is lower than the upper limit of the value range of the ceramide content ratio characterizing the healthy group or the lower limit of the value range of the ceramide content ratio characterizing the non-healthy group, the test subject is evaluated as “being healthy”, and
when the ceramide content ratio thus calculated is higher than or equal to the upper limit of the value range of the ceramide content ratio characterizing the healthy group or the value or the lower limit of the value range of the ceramide content ratio characterizing the non-healthy group, the test subject is evaluated as “having a possibility of being non-healthy” or “having a high possibility of being non-healthy”.

13. The method according to claim 2, wherein in a case in which the average value of the ceramide content ratio of the healthy group is higher than the average value of the ceramide content ratio of the non-healthy group,
when the ceramide content ratio thus calculated is higher than the lower limit of the value range of the ceramide content ratio characterizing the healthy group or the upper limit of the value range of the ceramide content ratio characterizing the non-healthy group, the test subject is evaluated as “being healthy”, and
when the ceramide content ratio thus calculated is lower than or equal to the lower limit of the value range of the ceramide content ratio characterizing the healthy group or the upper limit of the value range of the ceramide content ratio characterizing the non-healthy group, the test subject is evaluated as “having a possibility of being non-healthy” or “having a high possibility of being non-healthy”; and
wherein in a case in which the average value of the ceramide content ratio of the healthy group is lower than the average value of the ceramide content ratio of the non-healthy group,
when the ceramide content ratio thus calculated is lower than the upper limit of the value range of the ceramide content ratio characterizing the healthy group or the lower limit of the value range of the ceramide content ratio characterizing the non-healthy group, the test subject is evaluated as “being healthy”, and
when the ceramide content ratio thus calculated is higher than or equal to the upper limit of the value range of the ceramide content ratio characterizing the healthy group or the value or the lower limit of the value range of the ceramide content ratio characterizing the non-healthy group, the test subject is evaluated as “having a possibility of being non-healthy” or “having a high possibility of being non-healthy”.

14. An apparatus for evaluating the health of skin, comprising:
a quantitative determination means for quantitatively determining respective contents of a non-hydroxyacyl-phytosphingosine ceramide component and a non-hydroxyacyl-sphingosine ceramide component included in a lipid sample prepared from a collected sample of a stratum corneum; and
an arithmetic means for calculating the content ratio of the quantitatively determined content of the non-hydroxyacyl-phytosphingosine ceramide component to the quantitatively determined content of the non-hydroxyacyl-sphingosine ceramide component, and evaluating the skin condition for skin disease of a test subject from the calculated content ratio.

15. An apparatus for evaluating the health of skin, comprising:
a quantitative determination means for quantitatively determining respective contents of one kind of ceramide component A and one kind of ceramide component B included in a lipid sample prepared from a collected sample of a stratum corneum; and
an arithmetic means for calculating the content ratio of the quantitatively determined content of the ceramide component A to the quantitatively determined content of the ceramide component B, and evaluating the health of the skin of a test subject from the calculated content ratio;
wherein the ceramide component A is selected from the group consisting of a non-hydroxyacyl-phytosphingosine ceramide component, a non-hydroxyacyl-6-hydroxysphingosine ceramide component, an esterified ω-hydroxyacyl-6-hydroxysphingosine ceramide component, and an esterified ω-hydroxyacyl-phytosphingosine ceramide component; and the ceramide component B is selected from the group consisting of a non-hydroxyacyl-sphingosine ceramide component and an α-hydroxyacyl-sphingosine ceramide component; provided that the case of selecting the non-hydroxyacyl-phytosphingosine ceramide component as the ceramide component A and the non-hydroxyacyl-sphingosine ceramide component as the ceramide component B is excluded.

16. The apparatus according to claim 14,
wherein the apparatus stores a database that associates the information of the ratio of the content of the non-hydroxyacyl-phytosphingosine ceramide component, to the content of the non-hydroxyacyl-sphingosine ceramide component, with the health condition of the skin; and
wherein the health of the skin is evaluated from the ratio of the content of the non-hydroxyacyl-phytosphingosine ceramide component to the content of the non-hydroxyacyl-sphingosine ceramide component calculated by the arithmetic means based on the association function of the database.

17. The apparatus according to claim 14, wherein the presence or absence of the onset of atopic dermatitis or psoriasis, the possibility of the onset of atopic dermatitis or psoriasis, the prophylactic state of atopic dermatitis or psoriasis, the progress of atopic dermatitis or psoriasis, the presence or absence of a tendency (predisposition) toward atopic dermatitis or psoriasis, the status of healing of atopic dermatitis or psoriasis, or therapeutic effects for atopic dermatitis or psoriasis, is evaluated as the skin health.

18. (canceled)

19. The apparatus according to claim 15, wherein the presence or absence of the onset of atopic dermatitis or psoriasis, the possibility of the onset of atopic dermatitis or psoriasis, the prophylactic state of atopic dermatitis or psoriasis, the progress of atopic dermatitis or psoriasis, the presence or absence of a tendency (predisposition) toward atopic dermatitis or psoriasis, the status of healing of atopic dermatitis or psoriasis, or therapeutic effects for atopic dermatitis or psoriasis, or at least one selected from the group consisting of the skin barrier function, the horny layer water content, the skin color or skin luster, the skin texture, and the desquamation of skin is evaluated as the health of skin.

20. The apparatus according to claim 14, wherein in a case in which the average value of the ceramide content ratio of the healthy group is higher than the average value of the ceramide content ratio of the non-healthy group,
when the ceramide content ratio thus calculated is higher than the lower limit of the value range of the ceramide content ratio characterizing the healthy group or the upper limit of the value range of the ceramide content ratio characterizing the non-healthy group, the test subject is evaluated as “being healthy”, and
when the ceramide content ratio thus calculated is lower than or equal to the lower limit of the value range of the ceramide content ratio characterizing the healthy group or the upper limit of the value range of the ceramide content ratio characterizing the non-healthy group, the test subject is evaluated as “having a possibility of being non-healthy” or “having a high possibility of being non-healthy”; and
wherein in a case in which the average value of the ceramide content ratio of the healthy group is lower than the average value of the ceramide content ratio of the non-healthy group,
when the ceramide content ratio thus calculated is lower than the upper limit of the value range of the ceramide content ratio characterizing the healthy group or the lower limit of the value range of the ceramide content ratio characterizing the non-healthy group, the test subject is evaluated as “being healthy”, and
when the ceramide content ratio thus calculated is higher than or equal to the upper limit of the value range of the ceramide content ratio characterizing the healthy group or the value or the lower limit of the value range of the ceramide content ratio characterizing the non-healthy group, the test subject is evaluated as “having a possibility of being non-healthy” or “having a high possibility of being non-healthy”.

21. The apparatus according to claim 15, wherein in a case in which the average value of the ceramide content ratio of the healthy group is higher than the average value of the ceramide content ratio of the non-healthy group,
when the ceramide content ratio thus calculated is higher than the lower limit of the value range of the ceramide content ratio characterizing the healthy group or the upper limit of the value range of the ceramide content ratio characterizing the non-healthy group, the test subject is evaluated as “being healthy”, and
when the ceramide content ratio thus calculated is lower than or equal to the lower limit of the value range of the ceramide content ratio characterizing the healthy group or the upper limit of the value range of the ceramide content ratio characterizing the non-healthy group, the test subject is evaluated as “having a possibility of being non-healthy” or “having a high possibility of being non-healthy”; and
wherein in a case in which the average value of the ceramide content ratio of the healthy group is lower than the average value of the ceramide content ratio of the non-healthy group,
when the ceramide content ratio thus calculated is lower than the upper limit of the value range of the ceramide content ratio characterizing the healthy group or the lower limit of the value range of the ceramide content ratio characterizing the non-healthy group, the test subject is evaluated as “being healthy”, and
when the ceramide content ratio thus calculated is higher than or equal to the upper limit of the value range of the ceramide content ratio characterizing the healthy group or the value or the lower limit of the value range of the ceramide content ratio characterizing the non-healthy group, the test subject is evaluated as “having a possibility of being non-healthy” or “having a high possibility of being non-healthy”.

22. The method according to claim 2, wherein the lipid sample is a lipid sample prepared from a collected sample of the stratum corneum from a non-rash part of a test subject or from a healthy part of a test subject who has not developed skin disease.

23. The method according to claim 2, wherein the health of the skin is evaluated, based on the association function between the information of the ratio of the content of one kind of ceramide component A selected from the group consisting the non-hydroxyacyl-phytosphingosine ceramide component, the non-hydroxyacyl-6-hydroxysphingosine ceramide component, the esterified-ω-hydroxyacyl-6-hydroxysphingosine ceramide component and the esterified-ω-hydroxyacyl-phytosphingosine ceramide component, to the content of one kind of ceramide component B selected from the group consisting of the non-hydroxyacyl-sphingosine ceramide component and the α-hydroxyacyl-sphingosine ceramide component, both ceramide components being included in the lipid sample prepared from a collected sample of a stratum corneum, and the health condition of the skin, from the ratio of the quantitatively determined content of the ceramide component A to the quantitatively determined content of the ceramide component B.

24. The apparatus according to claim 15, wherein the apparatus stores a database that associates the information of the ratio of the content of one kind of ceramide component A selected from the group consisting of the non-hydroxyacyl-phytosphingosine ceramide component, the non-hydroxyacyl-6-hydroxysphingosine ceramide component, the esterified-ω-hydroxyacyl-6-hydroxysphingosine ceramide component and the esterified-ω-hydroxyacyl-phytosphingosine ceramide component, to the content of one kind of ceramide component B selected from the group consisting of the non-hydroxyacyl-sphingosine ceramide component and the α-hydroxyacyl-sphingosine ceramide component, with the health condition of the skin; and
wherein the health of the skin is evaluated from the ratio of the content of ceramide component A to the content of ceramide component B calculated by the arithmetic means based on the association function of the database.
